(19) United States
(12) Patent Application Publication
(10) Pub. No.: US 2011/0077702 A1
(43) Pub. Date: Mar. 31, 2011

(54) Means and Methods for Influencing Electrical Activity of Cells

(75) Inventors: Gerard J.J. Boink, Amsterdam (NL); Hanno L. Tan, Amstelveen (NL); Jacques De Bakker Milo Thomas, Muiden (NL)

(73) Assignee: Academisch Medisch Centrum Bij de Universiteit van Amsterdam, Amsterdam (NL)

(21) Appl. No.: 12/891,172

(22) Filed: Sep. 27, 2010

Related U.S. Application Data
(63) Continuation-in-part of application No. PCT/NL2009/050153, filed on Mar. 27, 2009.

(30) Foreign Application Priority Data
Mar. 27, 2008 (EP) 08153443.0
Nov. 12, 2008 (EP) 08168931.7

(51) Int. Cl. A61N 1/362; A61N 1/00; C12N 5/02; C12N 15/63; A61K 31/7088; A61K 35/12

(52) U.S. Cl. 607/9; 607/115; 435/375; 435/320.1; 435/325; 514/44 R; 424/93.7; 514/44 A; 435/373

(57) ABSTRACT

The invention provides means and methods for providing a cell with a spontaneous electrical activity and means and methods for increasing the depolarization rate of a cell having a spontaneous electrical activity. Means and methods are provided comprising:

providing a cell with a compound capable of providing and/or increasing a pacemaker current I_{f}, and

diminishing electrical coupling between said cell and surrounding cells and/or reducing the inward rectifier current I_{K1} of said cell, and or increasing the availability of I_{Na} at depolarized potentials, preferably using overexpression of additional sodium channels.

### RELATED APPLICATIONS

This application is a continuation-in-part of PCT application No. PCT/NL2009/050153 designating the United States and filed Mar. 27, 2009; which claims the benefit of EP patent application number 08168931.7 and filed Nov. 12, 2008; which claims the benefit of EP patent application number 08153443.0 and filed Mar. 27, 2008 all of which are hereby incorporated by reference in their entireties.

### FIELD OF THE INVENTION

The invention relates to the fields of biology and medicine.

### BACKGROUND OF THE INVENTION

Various kinds of animal cells exhibit electrical activity. For instance, information is transferred by cells of the nervous system via electrical signals. Gastrointestinal motility involves electrical activity of gastrointestinal cells, whereas glucose-induced release of insulin involves electrical activity of pancreatic islet cells. Another important biological function involved with electrical signals is the heartbeat. The heartbeat is driven by action potentials (APs) generated spontaneously in the sinoatrial (SA) node. Old age and a variety of cardiovascular disorders may disrupt normal SA node function. This can result in disease-causing slow heart rates in conjunction with fast heart rates, called “sick sinus syndrome”. Due to aging of the general population and an associated rise in the prevalence of cardiovascular disease, the prevalence and clinical impact of this syndrome are likely to increase. Currently, cardiac rhythm disorders such as sick sinus syndrome (SSS) and AV nodal block (AVB) are usually treated by electronic pacemakers. Electronic pacemakers are of great value in the therapy of cardiac conduction disease. These devices have become more and more sophisticated over the past years, but there are shortcomings Items that need improvement include the lack of autonomic modulation of the heart rate, the limited battery life, unstable electrode position, and electronic or magnetic interference. Creating an autonomically controlled biological pacemaker would solve these limitations. As used herein, the term “biological pacemaker” is also referred to as “biopacemaker”.

Currently, bio-engineered pacemakers are experimentally combined with electronic pacemakers. Advantages of such combinations over electronic pacemakers comprise improved autonomic modulation and extended battery lives of the combined entity. For instance, patent application WO 2007/014134 describes a pacemaker system comprising an electronic pacemaker and a biological pacemaker, wherein the biological pacemaker comprises cells that functionally express a chimeric hyperpolarization-activated, cyclic nucleotide-gated (HCN) ion channel. However, this chimeric HCN channel exhibited bursts of tachyarrhythmias both in vitro and in vivo (Plotnikov A N et al. Heart Rhythm 2008). Proof of concept was obtained by implanting the tandem biological and electronic pacemaker combination in dogs (wild-type HCN2 and an engineered HCN2 mutant; the mutant demonstrated improved channel kinetics however with a lower level of gene expression). Pacemaker activity was measured, whereby both components complemented each other. When the biological component slowed, the electronic component took over. Subsequently, when the biological component slowed down in rate, the electronic component started to fire (while the electronic pacemaker did not fire if the rate of the biological component was fast enough). Hence, the electronic pacemaker component was needed in order to provide sufficient pacemaker function.

A drawback of this pacemaker combination is the fact that two separate entities have to be brought into a heart. Moreover, disadvantages involving limited battery life (although battery life was extended as compared to the use of an electronic pacemaker alone), unstable electrode position and electronic or magnetic interference are still present.

It would be advantageous to use a biological pacemaker only, since the above mentioned disadvantages of the electronic pacemaker component would be overcome and autonomic modulation would be possible. However, until now biological pacemakers do not provide sufficient heart function. Slow beating rates and periods of complete cessation of beating are observed. Furthermore, biological biopacemaker rhythms exhibited unexplained large variations in beating rates (cycle lengths) [Cai et al (2007); Bucchi et al (2006)].

It is an object of the present invention to provide means and methods for providing cells with spontaneous electrical activity and/or for increasing spontaneous electrical activity of cells having electrical activity, so that biological functions involving spontaneous electrical activity are provided, increased and/or restored. It is a further object of the present invention to provide improved biological pacemakers.

Accordingly, the present invention provides a method for providing a cell with a spontaneous electrical activity and/or increasing the depolarization rate of a cell having a spontaneous electrical activity, the method comprising:

providing a cell with a compound capable of providing and/or increasing a pacemaker current I_{f}, and

diminishing electrical coupling between said cell and surrounding cells and/or increasing the availability of I_{Na} at depolarized potentials of said cell and/or increasing the firing frequency of said cell by increasing intracellular cAMP and/or increasing the firing frequency of said cell by decreasing action potential duration.

In one particular embodiment, the inward rectifier current I_{K1} of said cell is also reduced.

Said availability of I_{Na} is preferably increased using overexpression of at least one additional sodium channel and/or functional equivalent thereof in said cell. In one embodiment, said cell is provided with at least one sodium channel subunit.

Spontaneous electrical activity of a cell is herein defined as a firing capability, involving a spontaneous (i.e., without the need of an external electrical trigger) alteration of a cell's membrane potential in time (hyperpolarization/depolarization), resulting in transmission of excitation between cells (firing).

As used herein, a cell having spontaneous electrical activity is called a pacemaker cell.

With a method according to the present invention, a cell is provided with a spontaneous electrical activity and/or the spontaneous electrical activity of a cell is enhanced. This way, biological functions involving spontaneous electrical activity of cells are obtained, improved and/or restored.

One important biological function that is improved with a method according to the present invention is heartbeat. Without limiting the scope of the invention, cardiac applications are discussed in more detail.

In the heart, the pacemaker current I_{f} is naturally found in cells of the SA node. The SA node is a heterogonous structure composed of specialized cardiomyocytes and a high level of connective tissue. The activity in this node is driven by a spontaneous change in the membrane potential, called the slow diastolic depolarization or phase 4 depolarization. This phase 4 depolarization results in the formation of action potentials, thereby triggering the contraction of the heart. A major current underlying this process is the “funny current” or I_{f}. A family of hyperpolarization-activated cyclic nucleotidegated (HCN) channels underlies this inward current. There are four HCN isoforms (HCN1, HCN2, HCN3 and HCN4) which are all expressed in the human heart, but expression levels vary among regions. The activity of HCN channels is controlled by the cyclic adenosine monophosphate (cAMP)-binding site which allows alteration of activation kinetics by beta-adrenergic and muscarinic stimulation. By this mechanism, channel activity is increased or decreased. This plays an important role in the autonomic regulation of heart rate.

In a method according to the invention, a cell is provided with a compound capable of providing and/or increasing a pacemaker current I_{f}. In one preferred embodiment said compound comprises a hyperpolarization-activated cyclic nucleotide-gated (HCN) channel or a functional equivalent thereof. HCN channels underlie the Iₕ current (termed also I_{f} in the heart and I_{q} in the brain). The most prominent function proposed for this current is the generation of spontaneous rhythmic activity in heart, brain and insulin-secreting cells. Therefore, I_{f} has been called ‘pacemaker current’ and HCN channels have been designated ‘pacemaker channels’. Increasing HCN current results in increased diastolic depolarization, thereby enhancing the basal firing frequency.

A HCN channel is a sodium/potassium cation channel that is activated by membrane hyperpolarization. Activation of HCN channels results in an inward current carried by sodium/potassium which causes depolarization of the membrane potential. Hence, administration of HCN to a cell provides said cell with a pacemaker current or increases the pacemaker current of said cell.

The isoforms of HCN are capable of forming heterotetrameric complexes. Moreover, it is possible to design a HCN channel which comprises components of at least two different HCN forms. Alternatively, or additionally, one or more components of a HCN channel is/are modified, added or deleted in order to obtain a HCN-derived cation channel. As used herein, the term HCN or functional equivalent thereof embraces such embodiments. A functional equivalent of a HCN channel is defined as a compound which has at least one same property as HCN in kind, not necessarily in amount. A functional equivalent of a HCN channel is capable of being activated by membrane hyperpolarization; this activation results in an inward current carried by sodium/potassium which causes depolarization of a membrane potential. A functional equivalent of a HCN channel is for instance formed by building a cation channel using elements of different HCN isoforms. For instance, HCN1 exhibits rapid activation kinetics, whereas HCN4 exhibits a stronger cAMP response. In one embodiment HCN1 and HCN4 elements are therefore combined in order to obtain a HCN channel with an improved combination of activation kinetics/cAMP response properties. In a preferred embodiment, a cell is provided with HCN2, or a functional equivalent thereof, in a method according to the invention because HCN2 exhibits a strong cAMP response. As is apparent from Example 5, overexpression of HCN2 in the heart is particularly suitable for providing biological pacemaker function. Therefore, HCN2 is particularly useful in a method of the present invention.

Alternatively, or additionally, a functional equivalent of HCN, preferably HCN2, comprises at least one modified HCN sequence, as compared to natural HCN. In one embodiment a functional equivalent of HCN is provided through amino acid deletion and/or substitution, whereby an amino acid residue is substituted by another residue, such that the overall functioning is not seriously affected. Preferably, however, a HCN channel is modified such that at least one property of the resulting compound is improved as compared to wild type HCN. In a preferred embodiment a HCN mutant is used which is designed to shift the I_{f} activation curve to depolarized potentials. Such shift results in I_{f} current being more easily activated, i.e., at a potential which lies more closely to the resting membrane potential. Said shift is preferably essentially similar to the shift upon cAMP stimulation which normally results from beta-adrenergic stimulation. In one embodiment, a cell is provided with a functional equivalent of HCN as well as with wild-type HCN. These strategies further increase inward currents and result in faster beating.

In one particularly preferred embodiment a compound capable of providing and/or increasing a pacemaker current comprises a nucleic acid sequence encoding at least one HCN channel or a functional equivalent thereof. As used herein, the term “nucleic acid” encompasses natural nucleic acid molecules, such as for instance DNA, RNA and mRNA, as well as artificial sequences such as for instance a DNA/RNA helix, peptide nucleic acid (PNA), locked nucleic acid (LNA), et cetera. Many methods are known in the art for providing a cell with a nucleic acid sequence. For instance, calcium phosphate transfection, DEAE-Dextran, electroporation or liposome-mediated transfection is used. Alternatively, direct injection of the nucleic acid is employed. Preferably however said nucleic acid is introduced into the cell by a vector, preferably a viral vector. Most preferably long-term expression vectors are used, such as for instance Adeno Associated Vectors (AAV) or retroviral vectors, such as a lentiviral vector. Although adenoviral vectors (Ad) efficiently transduce cells, their usefulness as a therapeutic tool is limited, because they mediate only transient gene expression. An advantage of lentiviral vectors is that they integrate into the host genome. This induces long-term transgene expression, and renders these vectors ideal candidates to manage a chronic condition, such as sick sinus syndrome. Lentiviral vectors, for instance derived from human immunodeficiency virus (HIV) are therefore preferred. AAV vectors have the advantage that they are better suitable to scale up production capacity for therapeutic applications, for instance. Cardiac specific AAV serotypes such as AAV-1, AAV-6, AAV-8 and AAV-9 are therefore also preferred.

Various terms are known in the art which refer to introduction of nucleic acid into a cell by a vector. Examples of such terms are “transduction”, “transfection” or “transformation”. Techniques for generating a vector with a nucleic acid sequence of interest and for introducing said vector into a cell are known in the art. If desired, it is possible to use marker genes in order to determine whether a nucleic acid of interest has been introduced into a cell, as is well known in the art. See for instance the well known handbook of Sambrook and Russell (Molecular cloning, a laboratory manual, third edition, 2001 Cold Spring Harbour Laboratory Press, Cold Spring Harbour, N.Y.).

One preferred embodiment thus provides a method according to the invention, wherein a cell is provided with a hyperpolarization-activated cyclic nucleotide-gated (HCN) channel or a functional equivalent thereof, or a nucleic acid sequence coding therefore. In one embodiment said nucleic acid sequence encodes a wild type HCN. In one preferred embodiment, however, said nucleic acid sequence encodes a HCN mutant which is designed to shift the I_{f} activation curve to depolarized potentials. As explained above, such shift results in I_{f} current being more easily activated, i.e., at a potential which lies more closely to the resting membrane potential. In another preferred embodiment, one or more nucleic acid sequence(s) encoding a HCN mutant and wild type HCN is/are used.

Another preferred embodiment provides a method according to the present invention, wherein the basal cAMP level within said cell is enhanced. An increase in intracellular cAMP levels directly shifts I_{f} activation towards more depolarized potentials and it also stimulates intracellular Ca²⁺ handling via PKA dependent phosphorylation of involved proteins, such as the L-type Calcium channel, SERCA end RyR. This increases beating rates. In one embodiment the basal cAMP level of a cell is enhanced by increasing the amount and/or activity of a cAMP producing enzyme within said cell. In this embodiment an increased amount of cAMP is produced by said enzyme, resulting in increased pacemaker activity. Said enzyme preferably comprises an adenylate cyclase (AC), more preferably adenylate cyclase-1 and/or adenylate cyclase-8. These enzymes are Ca/calmodulin stimulated ACs and provide a crucial link between I_{f} based impulse formation and spontaneous Ca²⁺ oscillations, a mechanism that also importantly contributes to normal SA node impulse formation. An increased amount and/or activity of AC therefore improves biopacemaker function.

The amount of a cAMP producing enzyme in a cell is increased using any method known in the art. For instance, a nucleic acid sequence encoding a cAMP producing enzyme, or a functional equivalent thereof which is also capable of increasing cellular cAMP levels, is introduced into said cell, for instance using a (viral) vector. Said nucleic acid sequence is preferably operably linked to a promoter. If desired, an inducible promoter is chosen, so that the amount of expression can be regulated at will. This is, however, not necessary. Of course, alternative methods known in the art for increasing an amount of an enzyme of interest are suitable as well. The choice for a certain method depends on the specific circumstances.

Various methods for increasing the activity of a cAMP producing enzyme are also known in the art. For instance an activator, or a nucleic acid sequence encoding an activator, is administered to a cell comprising said cAMP producing enzyme, resulting in an enhanced activity of said enzyme.

In another embodiment, the basal cAMP level within a cell is enhanced by reducing the amount and/or activity of an enzyme involved with cAMP breakdown. Said enzyme preferably comprises a phosphodiesterase (PDE). Tissue-specific enzyme subtypes of PDE appear to be involved in subcellular regulation of cAMP mediated by cAMP breakdown. In particular, PDE3 and PDE4 are involved in beta-adrenergic independent and dependent signalling, respectively. Both sarcolemmal ion channels (e.g., HCN) and sarcoplasmatic reticulum (SR) Ca²⁺ handling proteins (e.g., SERCA) are regulated by these enzymes. Suppression of PDE activity therefore improves biopacemaker function.

Various methods are known in the art for reducing the amount and/or activity of a given enzyme. For instance, an antisense nucleic acid sequence and/or siRNA is administered to a cell in order to suppress expression of said enzyme. It is also possible to add an enzyme inhibitor, or a nucleic acid sequence coding therefore. Increasing the amount and/or activity of such enzyme inhibitor thus indirectly decreases the amount and/or activity of said enzyme. A non-limiting example of a PDE inhibitor is PI3Kγ, which is an upstream modulator of PDE activity. Increasing the amount and/or activity of PI3Kγ results in a reduced amount and/or activity of PDE.

In one preferred embodiment a cell is provided with:

an siRNA and/or an antisense nucleotide sequence against a phosphodiesterase; and/or

a nucleic acid sequence or a functional equivalent thereof encoding a phosphodiesterase with a diminished function as compared to wild type phosphodiesterase. Such phosphodiesterase with a diminished function is less capable of inducing cAMP breakdown. It has preferably retained its capability of binding free cAMP, so that free cAMP is bound but not, or to a lesser extent, degraded. A cell is preferably provided with a nucleic acid sequence or a functional equivalent thereof encoding a phosphodiesterase with a diminished function as compared to wild type phosphodiesterase, wherein said nucleic acid sequence or functional equivalent thereof encodes a phosphodiesterase with a dominant diminished function as compared to wild type phosphodiesterase. Such phosphodiesterase is preferably capable of suppressing the activity of wild type phosphodiesterase, for instance by forming a complex with a wild type phosphodiesterase thereby at least in part inhibiting its activity.

I_{f} is not the only current contributing to the pacemaker cell membrane potential. Other inward and outward currents are involved as well. Any increase in inward (e.g. I_{Na} or I_{Ca}) and/or decrease in outward current (e.g. I_{K1}) initiates or accelerates the process of phase 4 depolarization. Inward and outward currents of a pacemaker cell are often promoted or blocked by electrical interactions with the surrounding tissue. Interfering with electrical coupling between a pacemaker cell and surrounding cells therefore significantly influences these inward and outward currents, and pacemaker activity.

In one preferred embodiment of the present invention a cell is provided with a compound capable of providing and/or increasing a pacemaker current I_{f}, and electrical coupling between said cell and surrounding cells is diminished. According to the present invention, partial electrical uncoupling, reducing the electrical load and/or physical uncoupling stabilizes the function of a pacemaker cell. As a consequence the required size of a pacemaker region is reduced. Paradoxically, the spread of electrical activation is improved by partial uncoupling. Without being bound to theory, an explanation for these findings lies within the effects of the inward rectifier current (I_{K1}) in the surrounding tissue, which acts to maintain the resting membrane potential, thereby counteracting depolarization of the pacemaker region. Partial uncoupling alleviates this load-mismatch, because it isolates the small region of pacemaker cells from these effects of I_{K1} from the large surrounding region. This reduces the amount of pacemaker current which is required for successful generation of spontaneous action potentials in the pacemaker region and subsequent spread of activation to the surrounding regions.

One embodiment of the present invention provides a method for providing a cell with a spontaneous electrical activity and/or increasing the depolarization rate of a cell having spontaneous electrical activity, wherein the electrical coupling between said cell and surrounding cells is diminished by providing a barrier between said cell and surrounding cells. Said barrier preferably comprises a cell with a reduced conductor capacity as compared to the same kind of cell in a natural situation, so that the electrical coupling between a pacemaker cell and the surrounding region is diminished. In one embodiment, said barrier comprises fibrotic cells. As used herein, a fibrotic cell is defined as an injured cell that has a lower capability to conduct or generate an electrical impulse as compared to normal, healthy cells. Preferably, said fibrotic cell has lost its capability to conduct or generate an electrical impulse. Fibrotic cells are obtained in various ways. Preferably, surrounding cells are rendered fibrotic by heating them with a heating element with a temperature of at least 55° C., preferably at least 60° C. or cooling them with a cooling element with a temperature of at most −75° C., preferably at most −80° C. One embodiment therefore provides a method according to the invention for providing a cell with a spontaneous electrical activity and/or increasing the depolarization rate of a cell having spontaneous electrical activity, wherein the electrical coupling between said cell and surrounding cells is diminished by heating surrounding cells with a device having a temperature of at least 55° C. Said cells are preferably heated with a device having a temperature of about 60° C. Preferably, said surrounding cells are heated with a device having a temperature of between 50° C. and 75° C., more preferably between 50° C. and 65° C. In one particularly preferred embodiment said surrounding cells are heated with a device having a temperature of between 55° C. and 60° C. In one preferred embodiment (catheter-based) radiofrequency ablation is used. With this technique, a targeted area is gently warmed to about 60° C. to completely disrupt cell-to-cell electrical connections. A non-limiting example of impulse protection based on physical uncoupling in combination with partial uncoupling or load reduction is schematically depicted in FIG. 1.

Further provided is therefore a method according to the invention, wherein said barrier comprises cells which have been heated with a device having a temperature of at least 50° C., preferably with a device having a temperature of between 50 and 65° C., more preferably with a device having a temperature of between 55 and 60° C.

Yet another embodiment provides a method according to the invention for providing a cell with a spontaneous electrical activity and/or increasing the depolarization rate of a cell having spontaneous electrical activity, wherein the electrical coupling between said cell and surrounding cells is diminished by cooling surrounding cells, preferably to about −80° C. In one preferred embodiment cryo ablation is used. With this technique, a targeted area is gently cooled, preferably to about −80 ° C., to disrupt cell-to-cell electrical connections.

In one particularly preferred embodiment the electrical coupling between a pacemaker cell and surrounding cells is diminished such that the electrical impulse of a firing pacemaker cell will not, or to a significantly lesser extent, be conducted into a certain direction. Hence, preferably, electrical connections between a pacemaker cell and surrounding tissue are primarily present in one or several directions, whereas electrical impulses to at least one other direction are preferably diminished. This is for instance performed by providing a conductor barrier which partly surrounds said pacemaker cell. Said barrier preferably has a shape that allows for diminishing, preferably blocking, electrical connections between said cell and the surrounding tissues in at least one direction. In a particularly preferred embodiment, electrical connections to the surrounding tissues are blocked in at least one direction with respect to the plane that runs parallel to the heart surface. A non-limiting example is schematically depicted in FIG. 1: an electrical impulse of firing pacemaker cells is conducted to a certain direction because other directions are blocked by a barrier (for instance fibrotic cells).

Another way of diminishing electrical coupling between a pacemaker cell and surrounding cells is reduction of the amount and/or activity of gap junction proteins connecting said cell and surrounding cells. A gap junction is a junction between cells that allows different molecules and ions, mostly small intracellular and intercellular signaling molecules (intracellular and intercellular mediators), to pass freely between cells. A gap junction comprises protein channels in cell membranes that allow ions and small molecules to pass between adjacent cells. The protein channels that make up gap junctions usually consist of two connexons. One connexon resides in the membrane of one cell. It aligns and joins the connexon of the neighboring cell, forming a continuous aqueous pathway by which ions and small molecules can freely pass (passively) from one cell to the other. Connexons usually consist of six subunits called connexins. The connexin genes have been highly conserved during evolution. In some cells the connexons are formed of six identical connexins or of some combination of two different connexins.

Reducing the amount and/or activity of gap junction proteins connecting a pacemaker cell and surrounding cells diminishes electrical coupling between said cells. Further provided is therefore a method according to the invention, wherein the electrical coupling between said cell and surrounding cells is diminished by reducing the amount and/or activity of gap junction proteins connecting said cell and surrounding cells. In ventricular myocardial tissue, the gap junction protein connexin 43 is highly expressed and in atrial myocardium both connexin 40 and 43 are abundantly present. In order to diminish electrical coupling between cardiac cells, a cardiac pacemaker cell is therefore preferably provided with a gap junction protein with a diminished conductor capacity as compared to connexin 43 and/or connexin 40. Since several connexins assemble together in order to form a connexon, gap junction proteins with a diminished conductor capacity will assemble with wild type connexins in a cell so that a connexon is formed which has a lower conductor capacity.

Further provided is therefore a method according to the invention, wherein the electrical coupling between a pacemaker cell and surrounding cells is diminished by providing said cell with a gap junction protein with a diminished conductor capacity as compared to connexin 43 and/or connexin 40.

In one embodiment the amount and/or activity of connexin 43 and/or connexin 40 of a pacemaker cell is reduced. A method according to the invention, wherein the electrical coupling between a pacemaker cell and surrounding cells is diminished by reducing the amount and/or activity of connexin 43 and/or connexin 40 of said cell is therefore also provided. The amount of connexin 43 and/or connexin 40 in a cell is for instance reduced using antisense nucleic acid and/or siRNA which is capable of reducing expression of connexins. The activity of connexin 43 and/or connexin 40 is for instance reduced by administration of connexins with a lower conductor capacity, or nucleic acid coding therefore, as described before.

One particularly preferred embodiment provides a method for providing a cell with a spontaneous electrical activity and/or increasing the depolarization rate of a cell having a spontaneous electrical activity, comprising providing said cell with a compound capable of providing and/or increasing a pacemaker current I_{f}, and providing said cell with:

an siRNA and/or an antisense nucleotide sequence against connexin 43; and/or

an siRNA and/or an antisense nucleotide sequence against connexin 40; and/or

a nucleic acid sequence or a functional equivalent thereof encoding a connexin with a lower conductor capacity than the conductor capacity of connexin 43; and/or

a nucleic acid sequence or a functional equivalent thereof encoding a connexin with a lower conductor capacity than the conductor capacity of connexin 40.

As used herein, a connexin with a lower conductor capacity than the conductor capacity of connexin 40 or connexin 43 is defined as a connexin or a functional equivalent thereof which is capable of assembling with other connexins in order to form a connexon through which ions and other small molecules can pass from one cell to the other, wherein less molecules are capable of passing through the resulting connexon within a given time frame as compared to a connexon which is solely composed of wild type connexins 40 and/or 43.

Non-limiting, preferred examples of connexins with a lower conductor capacity than the conductor capacity of connexin 40 or connexin 43 are connexin 30.2, connexin 45 and connexin43Δ, a mutated connexin 43 with dominant negative characteristics, for example as described by (Krutovskikh V A Molecular carcinogenesis 1998). Connexin 30.2 is an SA node-specific connexin. Further provided is therefore a method according to the invention, wherein the electrical coupling between a pacemaker cell and surrounding cells is diminished by providing said cell with connexin 30.2 and/or connexin 45 and/or connexin43Δ and/or a functional equivalent thereof.

In yet another embodiment a cell is provided with a spontaneous electrical activity and/or the depolarization rate of a cell having a spontaneous electrical activity is increased by providing said cell with a transcription factor capable of reducing connexin 43 expression and/or connexin 40 expression so that fewer connexons are formed. Said transcription factor preferably comprises TBX3.

Spontaneous electrical activity is also provided or enhanced by administration of a beta-subunit for a voltage gated potassium channel (e.g. MirP1) to a cell. Such beta-subunit is capable of forming a complex with HCN, thereby increasing the pacemaker current I_{f}. Preferably, a cell is provided with a nucleic acid sequence encoding said beta-subunit. Further provided is therefore a method according to the invention, further comprising providing a cell with a beta-subunit for a voltage gated potassium channel and/or a nucleic acid sequence or a functional equivalent thereof encoding a beta-subunit for a voltage gated potassium channel.

In one embodiment, spontaneous firing frequency is provided or enhanced by a reduction in action potential (AP) duration. A reduction in AP duration is preferably achieved by overexpressing at least one voltage gated potassium channel responsible for repolarisation. AP shortening is particularly efficient because of the fact that the AP is prolonged in depolarized biopacemaker cells. Non-limiting examples of voltage gated potassium channels are Kv1.1-3 (or the constitutive mutant Kv1.3 H401W), Kv1.4-10 and Kv4.1-3.

Preferably, a cell is provided with a nucleic acid sequence encoding said beta-subunit. Further provided is therefore a method according to the invention, further comprising providing a cell with at least one voltage gated potassium channel responsible for repolarisation and/or a nucleic acid sequence or a functional equivalent thereof encoding at least one voltage gated potassium channel responsible for repolarisation. Said voltage gated potassium channel responsible for repolarisation preferably comprises a voltage gated potassium channel selected from the group consisting of Kv1.1-3, Kv1.3 H401W, Kv1.4-10 and Kv4.1-4.3.

In one aspect of the invention, protection of impulse formation is achieved by a reduction in the electrical load imposed by cells that surround a pacemaker cell. Said load is preferably reduced by shifting the resting membrane potential of said surrounding cells to more positive potential. This will subsequently result in a shift to more positive potentials of the resting membrane potential (called the maximal diastolic potential, MDP) of a pacemaker cell. This enhances basal pacing rates, as the MDP is closer to the threshold potential at which the pacemaker AP is initiated, thereby stabilizing basal pacemaker firing rates. A direct load reduction is preferably achieved by reducing the repolarizing inward rectifier potassium current I_{K1}.

In one preferred embodiment of the present invention, the inward rectifier current I_{K1} of a pacemaker cell is preferably reduced by providing said pacemaker cell with

an siRNA and/or an antisense nucleotide sequence against an inwardly-rectifying channel; and/or

a nucleic acid sequence or a functional equivalent thereof encoding an inwardly-rectifying channel with a diminished function as compared to the same kind of inwardly-rectifying channel in a wild type form.

An siRNA and/or an antisense nucleotide sequence against an inwardly-rectifying channel is an siRNA and/or an antisense nucleotide sequence comprising a sequence which is complementary to a nucleic acid sequence encoding at least one protein of said inwardly-rectifying channel. Non-limiting examples of inwardly-rectifying channel are Kir 2.1, Kir2.2 and Kir3.1. When a cell has been provided with an siRNA and/or an antisense nucleotide sequence against such inwardly-rectifying channel, less proteins of said inwardly-rectifying channel will be expressed, resulting in a lower amount of inwardly-rectifying channels in said cell. This way, the inward rectifier current I_{K1} is reduced. In one particularly preferred embodiment said inwardly-rectifying channel comprises a Kir2.1 channel. Reducing the amount and/or activity of a Kir2.1 channel significantly reduces the inward rectifier current I_{K1}.

In yet another aspect of the invention, protection of impulse formation is achieved by increasing the sodium current of said cell. The availability of the early/fast sodium current is reduced in biopacemaker cells due to the reduced maximal diastolic potential as a direct result of HCN overexpression. According to one embodiment of the present invention, arrhythmogenic consequences and/or current-to-load mismatch problems that result from this reduced sodium current availability are counteracted by providing said cell with additional sodium channels and/or sodium channels with altered kinetics. Especially suitable is the skeletal muscle sodium channel encoded by SkM1, or a functional equivalent thereof (SCN4A or a constitutive active variant, such as for instance the mutant G1306E of SCN4A). Alpha (or beta) subunits of a sodium channel resulting in improved channel availability at depolarized potentials are also suitable. Further provided is therefore a method according to the invention, further comprising providing a cell with an additional sodium channel and/or sodium channel with altered kinetics. Preferably, said cell is provided with a nucleic acid sequence or a functional equivalent thereof encoding at least an alpha (and/or beta)-subunit of a voltage gated skeletal muscle sodium channel. As is apparent from Example 5 overexpression of SkM1 is particularly suitable for restoring the availability of the early/fast sodium current which is reduced in biopacemaker cells due to the reduced maximal diastolic potential as a result of HCN overexpression. Example 5 shows that HCN2/SkM1 overexpression in hearts completely eliminates dependence on the electronic pacemaker back-up pacemaker which was also implanted. This example shows that combination of HCN and SkM1 provides a particularly potent biopacemaker which performs better than any of the biological pacemaker strategies currently used in the art. The present inventors succeeded for the first time in providing biological pacemaker function with which biological pacemaker rhythms are generated in more then 95% of the time in large animals. Therefore, in a preferred embodiment a method according to the invention is provided wherein a cell is provided with an HCN channel, or a functional equivalent thereof, and with a SkM1 channel, or a functional equivalent thereof. Most preferably, said cell is provided with an HCN2 channel, or a functional equivalent thereof, and with a SkM1 channel, or a functional equivalent thereof.

In yet another aspect of the invention a test system is provided which is particularly suitable for studying different multiple-gene-therapy strategies in order to solve biopacemaker instabilities described in the art that result from current-to-load mismatch. In one embodiment, a test system according to the invention comprises a method for focal transduction of a sheet of excitable cells (monolayer), or patterned seeding of transduced cells and subsequent detection of electrical activity, for instance using electrodes (for instance silver electrodes), a multiple electrode array (MEA) or optical mapping. In such a system, focal transductions are preferably achieved with magnetically tagged vehicles (preferably lentiviruses) that are preferably applied locally and, preferably, for a relatively short period (such as for instance about 15 minutes). In one embodiment the vehicles are applied just on top of the monolayer (for instance using a Hamilton injection needle). Enhanced focal gene delivery is achieved by the application of a magnetic source (preferably a strong magnetic force), preferably positioned below the monolayer. The strength and size of the magnetic source determine the location and size of the transduced area (see FIG. 8A).

Alternatively, an in vitro cell sheet of pacemaker cells surrounded by non-pacemaker cells is obtained via methods of multiple and patterned seeding. In this embodiment, initial central seeding is preferably combined with transduction, preferably lentiviral transduction. The area of initial central seeding is preferably bordered by a ring or cylinder, which preferably comprises polysulfon or silicon. Said ring or cylinder determines the size of the pacemaker area. Surrounding of this central area, by non-pacemaker cells, is preferably achieved by a second seeding of freshly isolated myocytes after removal of the ring or cylinder (FIG. 8B).

Further provided is therefore a method for focal transduction of cells, preferably excitable cells, the method comprising providing said cells with magnetically tagged vehicles, which vehicles comprise a nucleic acid (or functional equivalent thereof) of interest, and applying a magnetic source in order to determine the location and size of the transduced area. Said vehicles preferably comprise viruses, more preferably lentiviruses.

Yet another embodiment provides a method for producing a system comprising pacemaker cells which are at least in part surrounded by non-pacemaker cells, the method comprising providing an area of pacemaker cells produced by a method according to the invention, said area being bordered by a composition, preferably a ring or cylinder, and subsequently removing the composition and at least in part surrounding the pacemaker area by non-pacemaker cells.

A method according to the invention is, amongst other things, particularly suitable for inducing and/or increasing spontaneous electrical activity in cardiac cells. This way, a biological pacemaker is provided, enabling stable long-term function at a physiological heart rate and enabling autonomic modulation, thereby circumventing regulation difficulties of electronic pacemakers. Further provided is therefore a method according to the invention for providing a cell with a spontaneous electrical activity and/or increasing the depolarization rate of a cell having a spontaneous electrical activity, wherein said cell is present in, or brought into, atrial or ventricular myocardium.

In one preferred embodiment said cell is present in, or brought into, an atrium of a heart. An electrical impulse of a cell that fires in this area of the heart is conducted via the atrioventricular node (AV node) to the ventricles of the heart. The AV node is capable of conducting a limited amount of electrical impulses. Hence, if the firing activity of cells in an atrium is too high and/or uncontrolled, the other parts of the heart are protected against an overload of electrical impulses, thereby avoiding heart rhythm disorders resulting in ventricular fibrillation or other lethal cardiac arrhythmias. This embodiment therefore provides an extra safety measure. Of course, this embodiment is only preferred if the AV node of a subject's heart functions properly. In one embodiment, an atrial biological pacemaker is generated by providing at least one atrial cell with a spontaneous electrical activity with a method according to the invention, and/or by increasing the depolarization rate of at least one atrial cell with a method according to the invention. Such atrial biological pacemaker is particularly suitable for treating sick sinus syndrome. Hence, the invention provides a method wherein a cardiovascular disorder, preferably sick sinus syndrome, is treated with an atrial biological pacemaker according to the invention. In one embodiment, an atrial biological pacemaker according to the invention is used without the use of an electronic pacemaker. This embodiment is particularly suitable for treating sick sinus syndrome.

If desired, however, an atrial biological pacemaker according to the invention is combined with an electronic pacemaker. Such combination is for instance suitable for treating AV nodal block, because in this case the electrical impulse of firing atrial cells will have difficulties in reaching the ventricles. A combination of a biological pacemaker according to the invention and an electronic pacemaker has improved properties as compared to the current experimental combinations of a biological pacemaker and an electronic pacemaker, amongst other things because the properties of a biological pacemaker according to the invention are improved as compared to conventional biological pacemakers. For instance, (autonomic modulation of) the heart rate is improved. Hence, one embodiment of the invention provides a method wherein a cardiovascular disorder, preferably AV nodal block, is treated with a combination of an atrial biological pacemaker according to the invention and an electronic pacemaker.

It is, of course, also possible to generate a ventricular biological pacemaker with a method according to the present invention. A ventricular biological pacemaker is generated by providing at least one ventricular cell (i.e., a cell located in the ventricular compartments, such as ventricular working myocardial cells or cells of the specialized conduction system) with a spontaneous electrical activity with a method according to the invention, and/or by increasing the depolarization rate of at least one ventricular cell with a method according to the invention. A ventricular biological pacemaker is for instance preferred when the AV node of a subject's heart does not function properly (or is at risk of not functioning properly). Hence, if a subject suffers from AV nodal block, a ventricular biological pacemaker according to the invention is preferred. The invention therefore provides a method wherein a cardiovascular disorder, preferably AV nodal block, is treated with a ventricular biological pacemaker according to the invention.

Also when the AV node of a subject's heart functions properly, the use of a ventricular biological pacemaker according to the present invention is advantageous because it provides an additional safety measure, since possible diminished function of the AV node in the future will not affect the biological pacemaker function.

In one embodiment, a ventricular biological pacemaker according to the invention is used without the use of an electronic pacemaker. If desired, however, a ventricular biological pacemaker according to the invention is combined with an electronic pacemaker. As described above, a combination of a biological pacemaker according to the invention and an electronic pacemaker has improved properties as compared to the current experimental combinations of a biological pacemaker and an electronic pacemaker, amongst other things because the properties of a biological pacemaker according to the invention are improved as compared to conventional biological pacemakers. For instance, (autonomic modulation of) the heart rate is improved. Hence, one embodiment of the invention provides a method wherein a cardiovascular disorder is treated with a combination of a ventricular biological pacemaker according to the invention and an electronic pacemaker.

A method according to the invention is suitable for providing or increasing spontaneous electrical activity in a cell of interest. In one embodiment gene therapy is applied wherein a cell of a subject, such as for instance a cardiac cell, is modified. This is for instance performed by providing a cell of a subject, preferably a cardiac cell, with a gene delivery vehicle or a vector according to the invention, preferably a (lenti)viral vector, which vector comprises at least one nucleic acid sequence for providing said cell with spontaneous electrical activity and/or for improving the spontaneous electrical activity of said cell. In another embodiment, however, a dysfunctional organ or tissue, such as for instance a subject's heart, is provided with a cell wherein spontaneous electrical activity has been provided or enhanced with a method according to the invention. Preferably, said cell comprises a stem cell or progenitor cell.

Stem cells provide an alternative delivery platform or pacemaker source, especially when upregulation or downregulation of multiple genes is desired to improve overall pacemaker function. It is possible to use undifferentiated stem cells as well as differentiated stem cells. For cardiovascular applications in human individuals, human mesenchymal stem cells (hMSCs, undifferentiated) and/or human cardiac myocyte progenitor cells (hCMPCs, either differentiated or undifferentiated) are preferably used. As used herein, the term “stem cell” also encompasses progenitor cells. Ex vivo gene transfer provides an efficient strategy to introduce at least one nucleic acid sequence which allows for the creation of a homogeneous stem cell population with optimal pacemaker characteristics. If needed, multiple genes are easily introduced into stem cells. The risk of immunogenic rejection is maximally reduced by the use of autologous cells. Preferably, ex vivo lentiviral gene transfer is used because lentiviral vectors efficiently transduce cells and integrate into the host genome, allowing stable, long-term transgene expression.

Undifferentiated stem cells are easier to culture and to expand, and they are also immunoprivileged. However, these cells only function as a delivery system (for instance of an inward pacemaker current, as described hereinbefore). These cells lack the complete set of ion channels involved in membrane hyperpolarization and generation of cardiac action potentials (APs). Connexin proteins, importantly involved in electrical coupling and cell-to-cell transmission of electrical impulse, are therefore preferably present in both achieving the required membrane hyperpolarization (to activate the HCN channels) and for the initiation of APs in adjacent quiescent cells. For this reason, if undifferentiated cells are used, suppression of connexin function or suppression of load (as discussed hereinbefore) will not only reduce I_{K1} effects, but it will also reduce HCN activation. Suppression of connexin function or suppression of load (as discussed hereinbefore) is therefore not directly compatible with undifferentiated cells. However, undifferentiated cells provide an optimal tool to deliver I_{f} currents, possibly combined with increased intracellular cAMP, and, as such, they are preferably injected alone or in combination with the injection of gene therapy vectors or differentiated stem cells.

Differentiated stem cells, while being more difficult to culture and expand, are better capable of incorporating the various pacemaker properties. Various Ca²⁺ handling proteins (e.g., RyR2, SERCA2a,b,c and NCX-1) are efficiently upregulated in the differentiation process with 5′-azacytidine and TGF-β1 (TGF-beta 1), whereas incorporation of some of these genes in a gene therapy vector is hampered by their relatively large size. Biopacemaker properties are ultimately tailored by optimized differentiation towards a spontaneously active pacemaker phenotype, preferably in combination with additional gene transfer to increase HCN currents and/or increase intracellular cAMP. Additionally, TBX3 overexpression is used to stimulate differentiation into a nodal phenotype and prevent further differentiation into a more mature, working myocardium, phenotype.

The invention furthermore provides a gene delivery vehicle or a vector or an isolated cell comprising a compound capable of providing and/or increasing a pacemaker current I_{f}, and a compound capable of diminishing electrical coupling between said cell and surrounding cells. A gene delivery vehicle or a vector or an isolated cell comprising a compound capable of providing and/or increasing a pacemaker current I_{f} and a compound capable of reducing the inward rectifier current I_{K1} of said cell is also herewith provided. As explained before, said compound capable of providing and/or increasing a pacemaker current I_{f} preferably comprises a hyperpolarization-activated cyclic nucleotide-gated (HCN) channel or a nucleic acid sequence coding therefore. Other preferred compounds capable of providing and/or increasing a pacemaker current I_{f} are:
a cAMP producing enzyme, an adenylate cyclase, adenylate cyclase-1, adenylate cyclase-8, a compound capable of increasing the amount and/or activity of a cAMP producing enzyme, a compound capable of reducing the amount and/or activity of an enzyme involved with cAMP breakdown, a phosphodiesterase with a diminished function as compared to wild type phosphodiesterase;
a nucleic acid sequence encoding at least one of the abovementioned compounds; and
an siRNA and/or antisense nucleotide sequence against a phosphodiesterase.

One embodiment of the present invention therefore provides a gene delivery vehicle or a vector or an isolated cell comprising:

a nucleic acid sequence or a functional equivalent thereof encoding a hyperpolarization-activated cyclic nucleotide-gated (HCN) channel, and

an siRNA and/or antisense nucleotide sequence against a phosphodiesterase.

Another embodiment provides a gene delivery vehicle or a vector or an isolated cell comprising:

a nucleic acid sequence or a functional equivalent thereof encoding a hyperpolarization-activated cyclic nucleotide-gated (HCN) channel, and

a nucleic acid sequence or a functional equivalent thereof encoding a compound selected from the group consisting of:

a cAMP producing enzyme, an adenylate cyclase, adenylate cyclase-1, adenylate cyclase-8, a compound capable of increasing the amount and/or activity of a cAMP producing enzyme, a compound capable of reducing the amount and/or activity of an enzyme involved with cAMP breakdown, a phosphodiesterase with a diminished function as compared to wild type phosphodiesterase.

Preferred compounds capable of diminishing electrical coupling between a pacemaker cell and surrounding cells are, as described hereinbefore:
a compound capable of reducing the amount and/or activity of gap junction proteins connecting said cell and surrounding cells, a gap junction protein with a diminished conductor capacity as compared to connexin 43, a gap junction protein with a diminished conductor capacity as compared to connexin 40, a compound capable of reducing the amount and/or activity of connexin 43 of said cell, a compound capable of reducing the amount and/or activity of connexin 40 of said cell, a connexin with a lower conductor capacity than the conductor capacity of connexin 43, a connexin with a lower conductor capacity than the conductor capacity of connexin 40; connexin 30.2, connexin 45, connexin 43Δ or a functional equivalent thereof, a transcription factor capable of reducing connexin 43 expression, a transcription factor capable of reducing connexin 40 expression, TBX3 or a functional equivalent thereof;
a nucleic acid sequence encoding at least one of the abovementioned compounds; and
an siRNA and/or antisense nucleotide sequence against connexin 43, an siRNA and/or antisense nucleotide sequence against connexin 40

Preferred embodiments of the present invention therefore provide a gene delivery vehicle or a vector or an isolated cell comprising:

a nucleic acid sequence or a functional equivalent thereof encoding a hyperpolarization-activated cyclic nucleotide-gated (HCN) channel, and

an siRNA against connexin 43, and/or an antisense nucleotide sequence against connexin 43, and/or an siRNA against connexin 40, and/or an antisense nucleotide sequence against connexin 40, and/or a nucleic acid sequence or a functional equivalent thereof encoding a compound selected from the group consisting of:

a compound capable of reducing the amount and/or activity of gap junction proteins connecting said cell and surrounding cells, a gap junction protein with a diminished conductor capacity as compared to connexin 43, a gap junction protein with a diminished conductor capacity as compared to connexin 40, a compound capable of reducing the amount and/or activity of connexin 43 of said cell, a compound capable of reducing the amount and/or activity of connexin 40 of said cell, a connexin with a lower conductor capacity than the conductor capacity of connexin 43, a connexin with a lower conductor capacity than the conductor capacity of connexin 40, connexin 30.2, connexin 45, connexin 43Δ or a functional equivalent thereof, a transcription factor capable of reducing connexin 43 expression, a transcription factor capable of reducing connexin 40 expression, TBX3 or a functional equivalent thereof.

Furthermore, preferred compounds capable of reducing the inward rectifier current I_{K1} of a pacemaker cell are, as described hereinbefore:
an inwardly-rectifying potassium channel with a diminished function as compared to the same kind of inwardly-rectifying potassium channel in a wild type form, and a Kir2.1 channel or a functional equivalent thereof;
a nucleic acid sequence encoding at least one of the abovementioned compounds; and
an siRNA and/or antisense nucleotide sequence against an inwardly-rectifying channel.

A preferred embodiment of the present invention therefore provides a gene delivery vehicle or a vector or an isolated cell comprising:

a nucleic acid sequence or a functional equivalent thereof encoding a hyperpolarization-activated cyclic nucleotide-gated (HCN) channel, and

an siRNA and/or antisense nucleotide sequence against an inwardly-rectifying channel.

Another preferred embodiment provides a gene delivery vehicle or a vector or an isolated cell comprising:

a nucleic acid sequence or a functional equivalent thereof encoding a hyperpolarization-activated cyclic nucleotide-gated (HCN) channel, and

a nucleic acid sequence or a functional equivalent thereof encoding a compound selected from the group consisting of:

an inwardly-rectifying potassium channel with a diminished function as compared to the same kind of inwardly-rectifying potassium channel in a wild type form, and a Kir2.1 channel or a functional equivalent thereof.

In one preferred embodiment a cell according to the present invention comprises a myocardial cell. A method or a cell according to the invention, wherein said cell comprises a myocardial cell, is therefore also provided. In one embodiment said cell comprises a cardiac stem cell or cardiac progenitor cell. These embodiments are particularly suitable for cardiovascular applications.

A gene delivery vehicle is defined herein as any compound or composition capable of delivering a nucleic acid sequence of interest to a cell. Non-limiting examples of gene delivery vehicles, well known in the art, are plasmid delivery systems, virus like particles stable nucleic acid lipid particles, cholesterol conjugates, cationic delivery systems, peptide delivery systems, lipoplexes and liposomes. In one preferred embodiment, however, said gene delivery vehicle comprises a vector.

High titer vector production is important for final vector quality and a prerequisite for in vivo testing. However, transgenes are sometimes toxic when highly expressed in producer cells, which negatively influences viral titers. To circumvent this problem, cardiac specific (e.g. using the cardiac troponin T promotor) reversed expression cassettes are preferably constructed in the viral backbone when required (e.g. with HCN constructs). Such expression cassettes are particularly suitable for any method according to the present invention. Such expression cassette is also particularly suitable for providing a cardiac cell with one nucleic acid sequence of interest, for instance a nucleic acid sequence encoding a HCN or a functional part thereof. For instance, cells of the sinoatrial node are provided with a nucleic acid sequence encoding a HCN, or a functional part thereof, using a cardiac specific reversed expression cassette according to the invention, thereby counteracting sick sinus syndrome. One embodiment thus provides a gene delivery vehicle or a vector comprising a cardiac specific promoter and at least one nucleic acid sequence selected from the group consisting of

at least one nucleic acid encoding a compound capable of providing and/or increasing a pacemaker current I_{f}, and

at least one nucleic acid encoding a compound capable of diminishing electrical coupling between a cell and surrounding cells, and

at least one nucleic acid encoding a compound capable of increasing the availability of I_{Na} at depolarized potentials of a cell, preferably encoding a sodium channel and/or a functional equivalent of a sodium channel and/or a sodium channel with altered kinetics and/or an alpha subunit of a sodium channel and/or a beta-subunit of a sodium channel, and

at least one nucleic acid encoding a compound capable of increasing the firing frequency of a cell by increasing intracellular cAMP and/or by decreasing action potential duration. In one embodiment, said gene delivery vehicle or vector comprises at least two of the above mentioned nucleic acid sequences. Preferably, said gene delivery vehicle or vector comprises a nucleic acid sequence or a functional equivalent thereof encoding an HCN channel, preferably HCN2, and a nucleic acid sequence or functional equivalent thereof encoding SkM1. This allows the generation of an improved biopacemaker which provides all heart beats, without the need for an electronic pacemaker, as explained in more detail before.

A use of said gene delivery vehicle or vector in a method according to the present invention is also provided, as well as a use of said gene delivery vehicle or vector for the preparation of a medicament. Said gene delivery vehicle or vector is preferably used for the preparation of a medicament against a cardiac conduction disorder, preferably sick sinus syndrome, and/or AV nodal block. A gene delivery vehicle or a vector according to the invention for use as a medicament is therefore also provided.

A method according to the invention is particularly suitable for treating a subject suffering from, or at risk of suffering from, a disorder associated with impaired function of a cell with a spontaneous electrical activity. Restoring or improving spontaneous cellular electrical activity with a method according to the invention, and/or providing a cell with a spontaneous electrical activity with a method according to the invention, results in alleviation of the symptoms of said disease and/or at least partial treatment of said disease. Further provided is therefore a method for treating a subject suffering from, or at risk of suffering from, a disorder associated with impaired function of a cell with a spontaneous electrical activity, the method comprising:

providing a cell of said subject with spontaneous electrical activity with a method according to the invention, and/or

increasing the depolarization rate of a cell of said subject with a method according to the invention, and/or

administering to said subject a therapeutic amount of a gene delivery vehicle and/or vector and/or a cell according to the invention.

In a preferred embodiment said disorder associated with impaired function of a cell with a spontaneous electrical activity is a cardiovascular disorder. A biopacemaker is preferably provided with a method according to the present invention. Further provided is therefore a method for treating a subject suffering from, or at risk of suffering from, a cardiovascular disorder, the method comprising:

providing a myocardial cell of said subject with spontaneous electrical activity with a method according to the invention, and/or

increasing the depolarization rate of a myocardial cell of said subject with a method according to the invention, and/or

administering to said subject a therapeutic amount of a gene delivery vehicle and/or vector and/or a cell according to the invention. Said gene delivery vehicle and/or vector and/or cell is preferably administered to an atrium or a ventricle of the heart of said subject.

In one preferred embodiment said cardiovascular disorder comprises a cardiac conduction disorder, preferably sick sinus syndrome and/or AV nodal block.

Dose ranges of compounds, nucleic acid sequences, gene delivery vehicles, vectors and cells according to the invention to be used in the therapeutic applications as described herein are preferably designed on the basis of rising dose studies in the clinic in clinical trials for which rigorous protocol requirements exist. Typically, a dose of 0.1-3 ml 1*10⁸-1*10¹⁰ TU/ml is used with lentiviral vectors. In one embodiment a compound, nucleic acid sequence and/or cell according to the invention is combined with a pharmaceutically acceptable excipient, stabilizer, activator, carrier, permeator, propellant, desinfectant, diluent and/or preservative. Suitable excipients are commonly known in the art of pharmaceutical formulation and may be readily found and applied by the skilled artisan. A non-limiting example of a suitable excipient for instance comprises PBS.

A subject (preferably a human being) is provided with an effective amount of a compound, nucleic acid sequence, gene delivery vehicle, vector and/or cell according to the invention via any suitable route of administration. For instance, a (vector comprising a) nucleic acid is injected into cells of interest of a subject, for instance into myocardial cells of a subject. Preferably at least one parameter indicative of a disorder associated with impaired function of a cell with a spontaneous electrical activity, for instance a cardiovascular disorder, is determined before and after administration of a compound, nucleic acid sequence, gene delivery vehicle, vector and/or cell according to the invention, allowing determining whether or not treatment is successful. If desired, administration of further doses is repeated as often as necessary, preferably until the above mentioned at least one parameter is considered to be acceptable. One example of a suitable parameter is the heart rate at rest and during exercise and the presence or absence of arrhythmias, complaints or signs/symptoms of impaired cardiovascular function (e.g., reduced exercise capacity, heart failure, dizziness, syncope).

Another aspect of the present invention provides a device for increasing the depolarization rate of a cell or a group of cells having spontaneous electrical activity, and/or for providing a cell or a group of cells with spontaneous electrical activity, said device comprising:

means for providing a cell with a compound capable of providing and/or increasing a pacemaker current I_{f}, and

means for diminishing electrical coupling between said cell or group of cells and surrounding cells.

Such device is particularly suitable for performing a method according to the invention, wherein a cell or a group of cells is provided with a compound capable of providing and/or increasing a pacemaker current I_{f}, and wherein electrical coupling between said cell(s) and surrounding cells is diminished. This provides better results as compared to conventional methods. A device according to the invention preferably comprises a catheter. Said means for providing a cell with a compound capable of providing and/or increasing a pacemaker current I_{f}, and said means for diminishing electrical coupling between said cell and surrounding cells are preferably different from each other. In one embodiment, said means for diminishing electrical coupling between said cell and surrounding cells comprises a heating element. Said heating element preferably comprises an element for radiofrequency ablation, as described herein before. In another preferred embodiment, said means for diminishing electrical coupling between said cell and surrounding cells comprises a cooling element, preferably an element for cryo ablation. Said means for providing a cell with a compound capable of providing and/or increasing a pacemaker current I_{f} preferably comprises an element for injection of a nucleic acid sequence.

In a particularly preferred embodiment, a device according to the present invention comprises a catheter comprising a heating element or a cooling element, as well as an element for injection of a nucleic acid sequence. Such catheter is preferably used in a method according to the invention wherein a cell or a group of cells is provided with a compound capable of providing and/or increasing a pacemaker current I_{f}, and wherein electrical coupling between said cell(s) and surrounding cells is diminished.

A device according to the invention preferably comprises a heating element or a cooling element with a shape which enables limitation of electrical connections between a pacemaker cell and surrounding tissue in at least one direction. After use of such device electrical connections between a pacemaker cell and surrounding tissue are primarily present in one or several directions, whereas electrical impulses to at least one other direction are diminished. Preferably, electrical impulses to at least one other direction are blocked. This allows regulation of impulse conduction into one or several desired directions. A device according to the invention preferably has a shape in which electrical connections to the surrounding tissues are only present in a limited amount of directions with respect to the plane that runs parallel to the heart surface. This means that impulse conduction is limited and/or blocked in at least one direction. Further provided is therefore a device according to the invention, which has a shape that allows for diminishing, preferably blocking, electrical connections between said cell or group of cells and the surrounding tissues in at least one direction. As explained before, the electrical coupling between a pacemaker cell and surrounding cells is preferably diminished such that the electrical impulse of a firing pacemaker cell will be conducted into a certain direction. This is for instance performed by providing a conductor barrier which partly surrounds said pacemaker cell or group of pacemaker cells. A non-limiting example thereof is schematically depicted in FIG. 1: an electrical impulse of a firing pacemaker cell is conducted to a certain direction because other directions are blocked by a barrier (for instance fibrotic cells).

A method according to the invention involves the use of a compound capable of providing and/or increasing a pacemaker current I_{f}, together with a compound capable of diminishing electrical coupling between said cell and surrounding cells and/or a compound capable of reducing the inward rectifier current I_{K1} of said cell. Such combination of compounds is suitable for therapeutic purposes in order to counteract a disorder associated with impaired function of a cell with spontaneous electrical activity. Further provided is therefore a combination of:

a compound capable of providing and/or increasing a pacemaker current I_{f}, and

a compound capable of diminishing electrical coupling between said cell and surrounding cells and/or a compound capable of reducing the inward rectifier current I_{K1} of said cell,

for use as a medicament.

Also provided is a use of:

a compound capable of providing and/or increasing a pacemaker current I_{f}, and

a compound capable of diminishing electrical coupling between said cell and surrounding cells and/or a compound capable of reducing the inward rectifier current I_{K1} of said cell,

for the preparation of a medicament for preventing or counteracting a disorder associated with impaired function of a cell with spontaneous electrical activity.

Said combination is preferably used for the preparation of a medicament against as a cardiovascular disorder. One embodiment thus provides a use of:

a compound capable of providing and/or increasing a pacemaker current I_{f}, and

a compound capable of diminishing electrical coupling between said cell and surrounding cells and/or a compound capable of reducing the inward rectifier current I_{K1} of said cell,

for the preparation of a medicament for preventing or counteracting a cardiovascular disorder.

In one preferred embodiment said compound capable of diminishing electrical coupling between said cell and surrounding cells comprises a device according to the invention, as described herein before. Most preferably, a catheter comprising a heating element or a cooling element, as well as an element for injection of a nucleic acid sequence, is used.

In yet another preferred embodiment a combination or use according to the invention is provided, wherein said compound capable of diminishing electrical coupling between said cell and surrounding cells comprises an siRNA and/or antisense nucleotide sequence against connexin 43 and/or an siRNA and/or antisense nucleotide sequence against connexin 40 and/or a nucleic acid sequence encoding a compound selected from the group consisting of:

a compound capable of reducing the amount and/or activity of gap junction proteins connecting said cell and surrounding cells, a gap junction protein with a diminished conductor capacity as compared to connexin 43, a gap junction protein with a diminished conductor capacity as compared to connexin 40, a compound capable of reducing the amount and/or activity of connexin 43 of said cell, a compound capable of reducing the amount and/or activity of connexin 40 of said cell, a connexin with a lower conductor capacity than the conductor capacity of connexin 43, a connexin with a lower conductor capacity than the conductor capacity of connexin 40, connexin 30.2, connexin 45, connexin 43Δ or a functional equivalent thereof, a transcription factor capable of reducing connexin 43 expression, a transcription factor capable of reducing connexin 40 expression and TBX3 or a functional equivalent thereof.

Additionally, or alternatively, a combination or use according to the invention is provided wherein said compound capable of providing and/or increasing a pacemaker current I_{f} comprises an siRNA and/or antisense nucleotide sequence against a phosphodiesterase and/or a nucleic acid sequence encoding a compound selected from the group consisting of: a cAMP producing enzyme, an adenylate cyclase, adenylate cyclase-1, adenylate cyclase-8, a compound capable of increasing the amount and/or activity of a cAMP producing enzyme, a compound capable of reducing the amount and/or activity of an enzyme involved with cAMP breakdown, and a phosphodiesterase with a diminished function as compared to wild type phosphodiesterase.

Additionally, or alternatively, a combination or use according to the invention is provided wherein said compound capable of reducing the inward rectifier current I_{K1} of said cell comprises an siRNA and/or antisense nucleotide sequence against an inwardly-rectifying potassium channel and/or a nucleic acid sequence encoding a compound selected from the group consisting of: an inwardly-rectifying potassium channel with a diminished function as compared to the same kind of inwardly-rectifying potassium channel in a wild type form, and a Kir2.1 channel or a functional equivalent thereof.

Additionally, or alternatively, a combination or use according to the invention is provided wherein said cell is provided with a compound capable of increasing the sodium current availability at depolarized potentials of said cell. Said compound preferably comprises a nucleic acid sequence encoding a compound selected from the group consisting of: a sodium channel (preferably a voltage gated sodium channel), a skeletal muscle voltage gated sodium channel (preferably SkM1 and/or SCN4A), an alpha subunit from a sodium channel (preferably a voltage gated sodium channel), an alpha subunit from a skeletal muscle voltage gated sodium channel (preferably SkM1 and/or SCN4A) and a compound (e.g. beta subunit of a sodium channel) capable of increasing the amount of current available from the fast/early sodium current at depolarized potentials. In a preferred embodiment a cell is provided with a SkM1 channel, or a functional equivalent thereof, and with an HCN channel, or a functional equivalent thereof. As described above, HCN2/SkM1 overexpression in the heart results in biological pacemaker function which provides heart beats, without the need for an electronic pacemaker. This way, a biopacemaker has been provided for the first time with which biological pacemaker rhythms are generated in more then 95% of the time in a large animal model. Therefore, in a preferred embodiment a combination or use according to the invention is provided wherein said cell is provided with an HCN channel, or a functional equivalent thereof, and with a SkM1 channel, or a functional equivalent thereof. More preferably, a combination or use according to the invention is provided wherein said cell is provided with an HCN2 channel, or a functional equivalent thereof, and with a SkM1 channel, or a functional equivalent thereof.

Additionally, or alternatively, a combination or use according to the invention is provided wherein said cell is provided with at least one voltage gated potassium channel responsible for repolarisation and/or a nucleic acid sequence, or a functional equivalent thereof, encoding at least one voltage gated potassium channel responsible for repolarisation. Said voltage gated potassium channel responsible for repolarisation preferably comprises a voltage gated potassium channel selected from the group consisting of Kv1.1-3, Kv1.3 H401W, Kv1.4-10 and Kv4.1-4.3. In one embodiment, a combination or use according to the invention is provided wherein said cell is provided with a compound capable of increasing the voltage dependent potassium current of said cell. Said compound preferably comprises a nucleic acid sequence encoding a compound selected from the group consisting of an alpha subunit from a voltage gated potassium channel and a compound (preferably a beta subunit) capable of increasing the amount of current available.

A pharmaceutical composition, comprising a gene delivery vehicle and/or a vector and/or a cell according to the invention, is also provided herein. Said composition optionally comprises a pharmaceutically acceptable excipient, stabilizer, activator, carrier, propellant, desinfectant, diluent and/or preservative. Suitable excipients are commonly known in the art of pharmaceutical formulation and may be readily found and applied by the skilled artisan

The invention is further explained in the following examples. These examples do not limit the scope of the invention, but merely serve to clarify the invention.

### EXAMPLES

### Prior Art Biopacemakers

Research on biological pacemakers has so far mainly focused on proof-of-principle concepts. None of these concepts provided stable function at an acceptable heart rate.

### Improving Biopacemakers

To develop a clinically relevant biological pacemaker, stable long-term function at a physiological heart rate and incorporation of autonomic modulation are crucial. We started our research with lentiviral vectors in an effort to ensure long-term overexpression of HCN4. Novel strategies to improve this biopacemaker are also developed. These improvements center around two concepts: (1) improving impulse formation to enhance basal pacing rate in combination with tailored autonomic responsiveness and (2) protecting impulse formation to stabilize basal pacing rate. To unravel the most important contributors to biopacemaker function, we employ different strategies in parallel.

### Materials and Methods

### Construction and Production of Lentiviral Vectors

The cDNAs for human HCN4 (Alexander Scholten, Institut für Biologische Informationsverarbeitung, Forschungszentrum Jülich, Jülich, Germany) and rat Cx43_{—}130 to 136 deletion mutant (Vladimir Krutovskikh, International Agency for Research on Cancer, Lyon, France) were sub-cloned into the lentiviral vector plasmid, pRRL-cPPT-CMV-PRE-SIN.²⁴ These vectors were designated LV-HCN4 and LV-Cx43Δ. A control vector in which the CMV promoter drives GFP expression (LV-GFP) was described earlier.²⁴ Additional bicistronic vector plasmids were constructed with HCN4 and TBX3. In this vector, gene expression is controlled by a CMV promoter and transgene expression is linked to GFP expression by an internal ribosome entry site (IRES) from the encephalomyocarditis virus (EMCV). These vectors were designated LV-HCN4-GFP and LV-TBX3-GFP, respectively. Lentiviral vectors were generated by cotransfection of HEK293T cells, concentrated and titrated as described previously.²⁵ LV-HCN4 and LV-Cx43Δ was generated similarly and titrated by detecting transgene expression on transduced HeLa cells with immunohistochemistry.

### Construction, Staining and Testing of Mutant HCN4

The EVY367-9 amino acids in the S3-S4 region of human HCN4 were deleted in a pcDNA I expression vector using site directed mutagenesis. Presence of the deletion and lack of other DNA changes were confirmed by sequencing. EVY deleted HCN4 and wild type were transfected in HEK 293 cells using lipofectamin and analysed using immunohistochemistry. Cells were fixed with methanol:acetone (4:1) and washed with PBS supplemented with Tween20 (0.05%). Anti-HCN4 goat polyclonal IgG (Santa Cruz Biotechnology) was used as primary antibody and donkey anti-goat IgG conjugated with Alexa 568 (Molecular Probes) was used as secondary antibody. The cells were subsequently embedded with Vecta Shield® containing DAPI. The biophysical properties of EVY deleted HCN4 were studied with co-transfections of GFP using patch-clamp analyses and compared with wild type.

### Cell Isolation and Culture of Neonatal Rat Ventricular Cardiac Myocytes

Animal experiments were performed in accordance with the Guide for the Care and

Use of Laboratory Animals published by the National Institute of Health (NIH Publication No. 85-23, revised 1996), and approved by the institutional committee for animal experiments.

Six neonatal rats were sacrificed in one procedure as described previously.²⁶ Briefly, rats were decapitated after which a cardiotomy was performed. The atria were removed and the ventricles were minced. Tissue fragments were washed, using a Hanks' balanced salt solution (HBSS) without Ca²⁺ and Mg²⁺ supplemented with 20 units/100 ml penicillin and 20 μg/100 ml streptomycin. Five to six dissociations were performed for 15 minutes at 36.5° C. The dissociations were performed using HBSS without Ca²⁺ and Mg²⁺ containing 20 units/100 ml penicillin, 20 μg/100 ml streptomycin, 0.2% trypsin and 60 μg/ml pancreatin. The obtained dissociation solutions were centrifuged and cell pellets were resuspended in culture medium.

The neonatal rat ventricular myocytes were cultured in M199 containing (mM): 137 NaCl, 5.4 KCl, 1.3 CaCl₂, 0.8 MgSO₄, 4.2 NaHCO₃, 0.5 KH₂PO₄, 0.3 Na₂HPO₄, and supplemented with 20 units/100 ml penicillin, 20 μg/100 ml streptomycin, 2 μg/100 ml vitamin B₁₂ and either 5% or 10% neonatal calf serum (NCS), 10% NCS was used only on the first day of culturing the cells. These cells were cultured on collagen coated glass at 37° C. in 5% CO₂.

### Cardiac Myocytle Progenitor Cells

### Isolation, Differentiation, Transduction and Co-culture

Cardiac myocyte progenitor cells were isolated from human fetal hearts obtained after elected abortion with prior informed consent and approval of the ethical committee of the University Medical Center Utrecht. Hearts were isolated and perfused using a Langendorff perfusion setup. After digestion with collagenase and protease, CMPCs were isolated from the cardiac cell suspension using magnetic beads coated with a Sca-1 antibody. Cells were cultured on 0.1% gelatin coated material, using SP++ medium (EBM-2 with EGM-2 additives, mixed 1:3 with M199) supplemented with 10% FCS (Gibco), 10 ng/ml basic Fibroblast growth factor (bFGF), 5 ng/ml epithelial growth factor (EGF), 5 ng/ml insuline like growth factor (IGF-1) and 5 ng/ml hepatocyte growth factor (HGF). CMPCs were differentiated in Iscove's Modified Dulbecco's Medium/Ham's F-12 (1:1) (Gibco) supplemented with L-Glutamine (Gibco), 2% horse serum, non-essential amino acids, Insulin-Transferrin-Selenium supplement, and 10-4 M ascorbic acid (Sigma). First, CMPCs were exposed to 5 μM 5′-azacytidine for three days, followed with 1 ng/mL TGFβ1 every three days. For electrophysiological experiments, differentiated cultures were dissociated using collagenase and replated on gelatin coated coverslips in densities ranging from single cells to monolayers. Undifferentiated CMPCs were cultured in non-differentiating conditions for up to a maximum of 40 passages.

To study the interaction of non-differentiated CMPCs with cadiac myocytes, CMPCs were transduced in the presence of 8 μg/ml Polybrene (Sigma) with a GFP or HCN4-GFP lentivector at a multiplicity of infection (MOI) of 2. Transduced cells were used after 4 days for co-culture experiments. LV-HCN4-GFP and LV-GFP transduced CMPCs were seeded on top of 6 day-old NRCM monolayers, 7 days after the initiation of co-culture, spontaneous beating rates were assessed by counting the contractions during 1 minute. These cultures were superfused with Tyrode's solution (36±0.2° C.) containing (mmol/L): NaCl 140, KCl 5.4, CaCl₂ 1.8, MgCl₂ 1.0, glucose 5.5, HEPES 5.0; pH 7.4 (NaOH).

To obtain hybrid cultures of LV-HCN4-GFP transduced CMPCs cocultured and surrounded by neonatal cardiac myocytes, polysulfon rings/cylinders were used for seeding the transduced CMCPs in a central φ 4 mm area. Myocytes were subsequently seeded on top of, and surrounding the CMPCs, and cultured for 7 days before proceeding to optical mapping experiments (FIG. 8B-C).

### Single Cell Transduction and Electrophysiological Recordings

Neonatal rat cardiac myocytes and CMPCs were transduced with LV-HCN4-GFP, TBX3-GFP and LV-GFP at a MOI of 0.1, HEK 293 cells were transfected with both HCN4 and GFP or EVY deleted HCN4 and GFP. Single electrophysiological cell experiments were performed 7-10 days and 2 months after transducing NRCMs and CMPCs, respectively, or after the HEK 293 transfection. Myocytes were trypsinized during 30 seconds to prepare them for patch-clamping. By this procedure, cardiac myocytes lost their cell-to-cell connections, became less flattened (which facilitated the use of glass micropipettes), but remained attached to the coverslip.

Action potentials, I_{f} and membrane currents were recorded at 36±0.2° using the perforated patch-clamp technique (Axopatch 200B Clamp amplifier, Axon Instruments Inc.). Signals were low-pass filtered (cut-off frequency: 5-kHz) and digitized at 5-kHz. Series resistance was compensated by ≧80%, and potentials were corrected for the estimated 15-mV change in liquid-junction potential. For voltage control, data acquisition, and analysis, custom-made software was used. Superfusion solution contained (mM): 140 NaCl, 5.4 KCl, 1.8 CaCl₂, 1.0 MgCl₂, 5.5 glucose, 5 HEPES; pH 7.4 (NaOH). Pipettes (2-3 MΩ, borosilicate glass) were filled with solution containing (mM): 125 K-gluc, 20 KCl, 5 NaCl, 0.22 amphotericin-B, 10 HEPES; pH 7.2 (KOH).

I_{f} was characterized using custom voltage-clamp protocols modified from those published previously.^{14,27} For current-voltage (I-V) relationships and activation properties, I_{f} was measured as Cs⁺ sensitive (5 mM) current during 6-s hyperpolarizing steps (range −30 to −110 mV) from a holding potential of −30 mV. The hyperpolarizing step was followed by an 8-s step to −110 mV to record tail current, then a 0.5-s pulse to 40 mV to ensure full deactivation Tail current, plotted against test voltage, provided the activation-voltage relation; the latter was normalized by maximum conductance and fitted with the Boltzmann function I/Iₘₐₓ=A/{1.0+exp[(V_{1/2}-V)/k]} to determine the half-maximum activation voltage (V_{1/2}) and slope factor (k).

Net membrane current was characterized by 500-ms hyper- and depolarizing voltage-clamp steps from a holding potential of −40 mV every 2 s (for protocol, see FIG. 6A) Membrane currents were normalized to cell size.

### Monolayer Transductions and Electrophysiological Recordings

Cardiac myocyte monolayers were transduced at a MOI of 2.5 and 5 with LV-HCN4 and at a MOI of 5 with LV-GFP. Measurements were performed 14-21 days after the transduction. Extracellular electrograms were recorded at 34.0±0.1° C. using silver electrodes in a glass pipette (tip diameter 50 μm) containing 140 mM NaCl. Baseline signals were recorded for 85 seconds, thereafter cultures were exposed to 1 mM of the cAMP analogue dibutiryl-cyclic-adenosine-monophosphate (DBcAMP). Ten minutes later, electrograms were recorded again for 85 seconds. Signals were low-pass filtered (cut-off frequency: 400 Hz) and digitized at 2-kHz with a 24-bits resolution. Data acquisition was performed with modified ActiveTwo system without the input-amplifiers (BioSemi); data were analyzed using custom made software based on Matlab (Mathworks). After acquisition, data were digital high-pass filtered, to remove baseline drift. Mains interference was removed with a digital 50 Hz filter.

### Focal Transduction, Patterned Culturing and Optical Mapping

Monolayers of NRCMs were inspected, and those with defects or nonbeating cultures were rejected before transduction. Cells were transduced with lentiviral vectors, 4 days after initial seeding and optical mapping was performed 4 days after transduction. Focal transduction of the central area in the monolayer was obtained using lentiviral vectors complexed to magnetic nanoparticles (System Biosciences). These complexes were subsequently injected just above the monolayer (˜1 mm), above a strong magnetic field. To limit the transduction outside the central area, the virus was removed, and monolayers were washed, 15-30 minutes after initial application of the virus (FIG. 8A).

As an alternative method for obtaining monolayers of pacemaker cells surrounded by non-pacemaker cells, we employed a method of patterned seeding and transduction. For this method initial seeding was limited to a φ 4 mm central area on collagen coated coverslips using custom made polysulfon cylinders, 21 hours after seeding, monolayers were washed, polysulfon cylinders were removed and a freshly isolated myocytes were seeded (FIG. 8B). Similar polysulfon rings were used for seeding transduced CMCPs in a central area.

Myocyte or hybrid cultures were stained with 50 μmol/L di-8-ANEPPS (Molecular Probes) or 10 μmol/L di-4-ANEPPS (Molecular Probes) for 15 minutes. Optical recordings were made in a custom-made setup. Excitation light was delivered by 6 cyan (505 nm) high power Light Emitting Diodes (LEDs) filtered by a 505/30 nm band-pass filter. In addition, a single 505 nm excitation LED with a 505/30 nm band-pass filter is used via the dichroic mirror (560 nm). Emission fluorescence was high-pass filtered (600 nm) and measured with a photodiode array (PDA; Hamamatsu C4675-102). Data acquisition was performed with modified ActiveTwo system without the input-amplifiers (BioSemi; FIG. 8C); data were analyzed using custom made software based on Matlab (Mathworks).³⁰ After acquisition, data were digital filtered.

### In Vivo Tamoxifen Inducible TBX3 Overexpression

Animal care was in accordance with national and institutional guidelines.

The TBX3^{Cre} allele has been previously described.³¹ For age determination of the embryos, couples were put together overnight when the female was in estrus. On the next day, the female was inspected for a vaginal plug and the animals were separated. Noon was considered ED 0.5. Genomic DNA prepared from amnion or tail biopsies was used for genotyping by PCR, using primers specific for Cre and the wild-type allele. Animal care was in accordance with national and institutional guidelines.

CAG-CAT-TBX3^{31,32}, MerCreMer³³ and Z/EG³⁴ transgenic mice have been described previously. The transgenic mice were identified by PCR analysis using primers specific for CAT, Cre and GFP genes. MerCreMer (MCM) transgenic mice were bred with CAG-CAT-TBX3 (CT3)/Z/EG double transgenic mice to generate MCM-CT3, MCM-Z/EG double or MCM-CT3-Z/EG triple transgenic mice. These mice have no phenotype. Upon administration of tamoxifen (Sigma T5648, 1 mg, intraperitoneal injection, 4 days) MerCreMer is activated and causes recombination according to the Cre-1oxP system. CAT and lacZ are recombined out of the CT3 and Z/EG constructs respectively, which results in TBX3 and EGFP expression in all cardiomyocytes, because the MerCreMer gene is driven by a heart specific promoter (α-MHC). This way TBX3 over expression can be studied in adult mice. After 4 days of tamoxifen administration mice were sacrificed and the hearts were isolated. Expression of EGFP as a positive control of successful recombination and a marker for leakage of the system in non-tamoxifen treated animals was evaluated by fluorescent microscopy. The left atrial appendage and the apex were separated from the rest of the heart and all parts of the heart were snap frozen in liquid nitrogen quickly.

Total RNA was isolated from apex and left atrial appendices using the Nucleospin Kit (Machery-Nagel) according to the manufacturer's protocol. cDNA was made by reverse-transcription of 300 ng of total RNA using the SuperScript II system (Invitrogen). Expression of genes was assayed with quantitative real-time PCR using the Lightcycler 480 (Roche). qPCR data were analyzed with LinRegPCR (Ramakers et al, 2003) to determine the PCR efficiency values per sample. Starting concentrations were calculated per sample using the mean PCR efficiency per amplicon and the individual CT-values.³⁵ Gene expression data are presented normalized for GAPDH expression.

### In Vivo Lentiviral Gene Transfer

Animal care was in accordance with national and institutional guidelines.

Adult, male, Wistar rats were anesthetised using Isoflurane (2-3%), intubated and mechanically ventilated using a Harvard Infant ventilator. A minimal invasive approach to obtain access to the heart was used. First the abdomen was opened via a median laparotomy, the thorax was subsequently opened via a T-shaped incision through the diaphragm and 50 μl lentivectors (1*10⁹ TU/ml) were injected into the apical free wall of the left ventricle (FIG. 10A). After injection, thorax and abdomen were closed and animals were allowed to recover. Seven days after gene transfer, animals were anesthetised, and organs were fixated in vivo using 2-4% paraformaldehyde. Hearts were thereafter fixated in 4% paraformaldehyde (4 hours), 30% sucrose (16 hours), snap frozen using liquid nitrogen and stored at −80° C. Whole ventricles were cryosectioned and embedded in VectaShield containing DAPI. Cryosections were studied using fluorescence microscopy. Three-dimensional reconstructions were prepared using AMIRA software.

### Statistics

Results are expressed as mean±SEM. Group comparisons were made using a Student's t-test. The level of significance was set at P<0.05.

### Example 1

Increasing Intracellular cAMP and Constitutive HCN Mutants

Basal cAMP levels are almost tenfold higher in SA node cells as compared to atrial and ventricular myocytes. The increase in intracellular cAMP both stimulates Ca²⁺ handling proteins and shifts the I_{f} activation curve towards more depolarized potentials. Both changes increase beating rates. Increased intracellular cAMP levels are for instance achieved by overexpressing cAMP producing enzymes (adenylate cyclase, AC) or reducing the expression or function of enzymes responsible for cAMP breakdown (phosphodiesterases, PDEs). In particular, PDE4 appears to be involved in β-adrenergic signaling of both sarcolemmal ion channels (e.g., HCN) and sarcoplasmic reticulum (SR) Ca²⁺ handling proteins (e.g., SERCA). Genetic suppression of PDE activity (for instance with siRNAs or PI3Kγ; an upstream modulator of PDE activity) are therefore considered important strategies to improve biopacemaker function.

An attractive strategy to increase HCN currents is the construction and use of constitutive HCN mutants. These mutants are preferably constructed from human HCN2 or HCN4 since these isoforms are most sensitive to cAMP and therefore most relevant for clinical biopacemaker development. Of particular interest are mutants that generate more HCN current at physiological potentials. Mutants are therefore generated with a shifted V_{1/2} towards more depolarized potentials. In these mutants maintenance or increase in current density is crucial for functional improvements and this is therefore investigated carefully. HCN currents are also potentially increased via modification of the instantaneous current, the latter is possibly achieved by slowing or disrupting channels closure via specific mutations.

### Results

PDE4 inhibition using rolipram in combination with HCN4 overexpression demonstrates a rightward shift in the I_{f} activation curve in NRCMs (see FIG. 2A)

PDE4 inhibition with rolipram in itself also demonstrates a remarkable increase in spontaneous activity in single NRCMs (FIG. 2B)

EVY deleted HCN4 was generated via site directed mutagenesis.

Similar membrane expression of EVY deleted HCN4 compared to wild type HCN4 was demonstrated with immonolabelling of transfected HEK 293 cells (FIGS. 7A and B).

EVY deleted HCN4 did not result in the expected V_{1/2} shift as demonstrated by patch-clamp analysis. Potential biopacemaker improvements however result from significantly slowed activation kinetics which increases instantaneous currents (FIG. 7C).

### Example 2

### Fine Tuning of Nodal Properties in Engineered Stem Cells

In addition to the various gene therapy strategies described hereinbefore, stem cells provide an alternative delivery platform or pacemaker source, especially when upregulation or downregulation of multiple genes is required to improve overall pacemaker function. In our effort to improve impulse formation, we described strategies to increase HCN current and to increase intracellular cAMP. These strategies can be incorporated and further developed in engineered stem cells. We therefore designed a third strategy that combines ex vivo lentiviral gene transfer using undifferentiated stem cells (human cardiac myocyte progenitor cells, hCMPCs, or human mesenchymal stem cells, hMSCs) or differentiated stem cells (hCMPCs). Ex vivo lentiviral gene transfer provides an efficient strategy to introduce multiple genes which allows for the creation of a homogeneous stem cell population with optimal pacemaker characteristics. The risk of immunogenic rejection is maximally reduced by the use of autologous cells.

Undifferentiated stem cells are easier to culture and to expand, and they may also be immunoprivileged. However, these cells only function as a delivery system (of an inward pacemaker current). These cells lack the complete set of ion channels involved in membrane hyperpolarization and generation of cardiac action potentials (APs). Connexin proteins, importantly involved in electrical coupling and cell-to-cell transmission of the electrical impulse, are therefore important in both achieving the required membrane hyperpolarization (to activate the HCN channels) and for the initiation of APs in adjacent quiescent cells. For this reason, suppression of connexin function or suppression of load will not only reduce I_{K1} effects, but it will also reduce HCN activation and is therefore not directly compatible with undifferentiated cells. However, undifferentiated cells provide an optimal tool to deliver I_{f} currents and, possibly combined with increased intracellular cAMP, as such, they are preferably combined with the injection of gene therapy vectors or differentiated stem cells.

We have transduced undifferentiated hCMPCs with lentiviral vectors.

### Results

Undifferentiated hCMPCs are efficiently transduced by lentiviral vecors; at a multiplicity of infection (MOI) of 2 approximately 70% of the cells are transduced (FIG. 3A).

Undifferentiated hCMPCs transduced with LV-HCN4-GFP demonstrated stable I_{f} expression for more than 2 months (FIG. 3B).

Basic cell morphology remains intact after lentiviral overexpression of GFP, HCN4 or TBX3 (FIG. 3C, D, E).

Electrical coupling was studied using LV-HCN4-GFP and LV-GFP transduced CMPCs which were seeded on top of NRCM monolayers. Spontaneous beating rates were assessed 7 days after the initiation of co-culture, and were significantly faster in LV-HCN4-CMPC/NRCM co-cultures (85±14 bpm) than in LV-GFP-CMPC/NRCM co-cultures (32±6 bpm; P<0.05; FIG. 3F).

Impulse generation and action potential propagation were studied using voltage sensitive optical mapping. In LV-HCN4-CMPC/NRCM hybrid co-cultures, a central focus with clear phase 4 depolarization could be demonstrated (FIG. 3G). Neither central foci nor phase 4 depolarization were detected in control monolayers that contained only NRCMs.

### Example 3

### Protecting Impulse Formation to Stabilize the Biopacemaker Rate: Impulse Protecting Genes and Procedures

Currently constructed biopacemakers using engineered HCN mutants or very large amounts of HCN overexpressing hMSCs have failed to increase the biopacemaker frequency within the physiological range. This probably results from an intensified response to parasympathetic stimulation during rest, a failure to drive, or a combination of both. In the previous examples we provided solutions to problems that could result from an intensified response to parasympathetic stimulation. Here, we describe novel biopacemaker concepts to protect impulse formation and thereby provide solutions for problems that center around load-mismatch and a failure to drive. Protecting the biopacemaker area by partial electrical uncoupling, reducing the electrical load, and/or physical uncoupling stabilizes function and subsequently reduces the required size of the biopacmaker region.

Partial uncoupling is for instance achieved by overexpression of certain connexin isoforms (depending on the target region, e.g., Cx30.2, Cx40 or Cx45) or suppression of Cx43 (with dominant negative constructs [e.g., Cx43Δ], siRNA or transcription factor overexpression, e.g., TBX3).

We also describe an in vitro screening system to investigate various solutions to current-to-load mismatch based biopacemaker instabilities. This system uses neonatal rat cardiac myocyte monolayers with a designated central area of virally transduced myocytes or stem cells. Focal transductions are for instance achieved with lentiviral particles coupled to magnetic nanoparticles and strong magnets directing them to the centre of the monolayer. Patterned seeding of transduced myoctes or stem cells are for instance achieved with custom made polysulfon or silicon rings/cylinders. Both methods are used to test various gene combinations or stem cell modifications in a setting where the biopacemaker cells are surrounded by non-pacemaker cells (FIG. 8A-B); this setting is similar to the in vivo situation. Functional electrophysiological analysis of these engineered in vitro biopacemakers is performed using single electrode extracellular recordings (FIG. 9A), multiple electrode arrays (MEAs), or optical mapping (FIGS. 3G, 4A-C, 8B and 9B-C).

### Results

Transduced monolayers of neonatal rat cardiac myocytes demonstrate remarkably stable cycle lengths when HCN4 is heterogeneously expressed throughout the monolayer. We found a critical transduction efficiency for the maintenance of cycle length stability when these monolayers are challenged with DBcAMP (FIG. 9A). A transduction efficiency of 27%, achieved at a multiplicity of infection (MOI) of 5, was already sufficient to maintain stable cycle lengths.

Pronounced instabilities were seen in experiments in which we introduced hyperpolarizing load from non-pacemaker cells into our patterned seeding monolayer system. In this system, spread of electrical activity was measured using voltage-sensitive dyes in a custom-built optical mapping setup. Initiation of electrical activity and phase 4 depolarization were located at the central area, the site of HCN4 expression (FIG. 9B). In this setup we also detected clear cycle length irregularities and “on-off-switching” (FIG. 9C).

Monolayers engineered with central HCN4 expression demonstrate improved impulse formation in combination with Cx43Δ (FIG. 4)

Partial uncoupling with Cx43Δ probably also increases the susceptibility to re-entry arrhythmias. (FIG. 4C)

Inducible TBX3 expression initiates down regulation of both connexin 43 and connexin 40 in adult atrial myocardium (FIG. 5)

In TBX3 overexpressing NRCMs, we observed a reduced instantaneous current in the voltage range of I_{Ca,L} activation and a reduced steady-state current in the voltage range of I_{K1} activation (FIG. 6B)

In control NRCMs, we observed a large transient inward current by stepping back from very negative potential with characteristics of the Na⁺ current. This current was absent in TBX3 overexpression cells (FIG. 6C)

TBX3 overexpressing NRCMs adopt hallmark features of nodal cells, possibly due to a reduced ‘background’ K⁺ current, I_{K1} (FIG. 6D).

### Example 4

### In Vivo Small and Large Animal Experiments and Procedures

### Virus Production for In Vivo Studies

Plasmid DNA for virus production is prepared using endotoxin-free methods. All used vectors are titrated using genomic copy determination and functional titration, the ratio between these two titers provides a measure for the viral quality of a specific preparation. Functional titration is performed on HEK 293 T cells in the presence of DEAE-Dextran and assayed using quantitative PCR based methods. Accurate determination of functional titers is important for standardisation in experiments using multiple vectors and also for determination of the therapeutic window of different vector and transgene combinations.

High titer vector production is important for final vector quality and a prerequisite for in vivo testing. Transgenes are sometimes toxic when highly expressed in producer cells, which negatively influences viral titers. To circumvent this problem, cardiac specific (e.g. using the cardiac troponin T promotor) reversed expression cassettes are constructed in the viral backbone when required (e.g. with HCN constructs).

Cardiac myocyte transduction will be achieved by injecting one or multiple sites in close proximity (0.1-5 cm). A total volume of 0.05-10 ml will be injected depending on the injection site, transgenes and expression vector. In a final set of experiments animals will be injected with vectors that are produced under GLP compliance.

### Stem Cell Preparation for In Vivo Injections

Differentiated and undifferentiated stem cells are cultured and maintained under endotoxin-free conditions. If transduced cells are used the transduction can be performed a couple of days to weeks before stem cell transplantation. Alternatively a monoclonal stem cell population is obtained after transduction. Collagenase enzymes are used to obtain single cell suspensions ready for transplantation. In a final set of experiments animals will be injected with stem cells that are isolated, expanded, and, if required, transduced and/or differentiated under GLP compliance.

### Small Animal Studies

Initial testing of some strategies is performed in our adult rat model for focal gene transfer (FIG. 10A). This model is not used for extensive biopacemaker testing, but is very useful for proof-of-principle and biodistribution studies. Lentivirally transduced myocytes are for example easily reconstructed using GFP expression vectors, cryosectioning and fluorescence microscopy (FIG. 10B-C)

Functional studies are started 4-28 days after virus injection, depending on the used vector and transgene. Biopacemaker function is unleashed by temporarily or permanently disrupting the AV-node, e.g., using vagal stimulation, pharmacological interventions or RF ablation.

### Large Animal Studies

To establish that strategies derived from in vitro or small animal in vivo studies are effective in a large animal, we validate these studies in mini-pigs or dogs. These large animals are chosen because of the following reasons: (1) their heart rates are more similar to human heart rates than those of small animals (e.g. , rat), reflecting that their cardiac electrophysiology is more human-like, and facilitating in depth analysis of biopacemaker function; (2) they age relatively quickly, allowing for studies at senescence, similar to sick sinus syndrome patients; (3) their small size (e.g., compared to conventional pigs) makes them easy to handle, facilitating long-term studies.

### Large Animal Experimental Procedures

A biopacemaker is created by direct injection of the viral or stem cell delivery-vehicle into the atrium, conduction system or ventricle. All three sites are clinically relevant and are therefore studied.

An atrial biopacemaker is injected after thoracotomy, the sinus node will be localized with epicardial activation mapping, and subsequently ablated (RF ablation or excision). For initial efficacy studies, the biopacemaker delivery-vehicle is injected subepicardially into the left atrium (for easier distinction of the origin of atrial activation by ECG). An electronic pacemaker (AAI or DDD mode) is also implanted for the following: (1) to ensure survival of the animal during the period when transgene expression is too low to sustain viable heart rates (shortly after gene transfer), (2) to monitor heart rates online by using pacemakers with telemetry, and (3) by analyzing stored rhythms, we will study biopacemaker efficacy (number of electronically paced beats above lower rate) and safety (tachyarrhythmias). Animals are also studied in the free-running state and during exercise testing with telemetry and ECG. The pig or dog is sacrificed, and the heart is isolated for studies into safety, organ/tissue and cellular electrophysiology, at study end (6 months) or when biopacemaker function is lost or when serious adverse events occur.

Ventricular and bundle branch biopacemakers are injected via catheter-based methods. In these studies the atrioventricular node will be destroyed using catheter-based RF ablation and an electronic pacemaker (VVI or DDD mode) will be implanted to serve as a back-up pacemaker and a monitoring device. Of special interest is the ventricular-apex injection site, since this could be an avenue of clear benefits of biological pacing. This pacing site potentially improves cardiac output in patients with bundle branch disease and it is difficult to approach for stable lead implantation with conventional electronic pacemakers.

### Example 5

Overexpression of SkM1 Enhances HCN2-based Biological Pacemaker Function

### Methods

Experiments were performed with the use of protocols approved by the Columbia University Institutional Animal Care and Use Committee and conform to the Guide for the Care and Use of Laboratory Animals (National Institutes of Health publication No. 85-23, revised 1996).

### Adenoviral Constructs

Adenoviral constructs of mouse (hyperpolarization-activated, cyclic nucleotide-gated) HCN2 driven by the CMV promoter and rat skeletal muscle Na⁺ channel SkM1 driven by the CMV promoter were prepared as previously described (14,47). For consistency with earlier studies (36), when samples were prepared for in vivo injection, 3×10¹⁰ fluorescent focus units of the HCN2 adenovirus was mixed with an equal amount of a green fluorescent protein (GFP)-expressing adenovirus or SkM1-expressing adenovirus, in a total volume of 700 μL.

### Intact Canine Studies

Adult mongrel dogs (Chestnut Ridge Kennels, Chippensburg, Pa.) weighing 22 to 25 kg were anesthetized with propofol 6 mg/kg IV and inhalational isoflurane (1.5% to 2.5%). With the use of a steerable catheter, HCN2/GFP (n=10) or HCN2/SkM1 (n=3) was injected into the left bundle branch as described previously (36). Complete atrioventricular (AV) block was induced via radiofrequency ablation, and each site of injection was paced via catheter electrode to distinguish electrocardiographically the origin of the idioventricular rhythm during the follow-up period. An electronic pacemaker (Guidant, Discovery II, Flextend lead, Guidant Corp, Indianapolis, Ind.) was implanted and set at VVI 35 bpm. ECG, 24-hour Holter monitoring, pacemaker log record check, and overdrive pacing at 80 bpm (or 5-10% faster then intrinsic rates) were performed daily for 8 days. For each dog, the percent electronic and percent biologically induced beats were calculated daily.

To evaluate β-adrenergic responsiveness at termination of the study, epinephrine (1.0 μg/kg per minute for 10 minutes) was infused and maximum rate response in the pace-mapped rhythm was recorded.

### Tissue Bath Studies

Preparations were placed in a 4-mL chamber perfused with Tyrode's solution (37° C., pH 7.3 to 7.4) at a rate of 12 mL/min and were permitted to beat spontaneously. Tyrode's solution containing Isoproterenol with/without Tetrodotoxin (TTX) or Ivabradine were freshly prepared on the day of the experiment. The bath was connected to ground via a 3 mol/L KCl/Ag/AgCl junction. Preparations were impaled with 3 mol/L KCl filled glass capillary microelectrodes that had tip resistances of 10 to 20 MΩ coupled by an Ag/AgCl junction to an amplifier with high input impedance and input capacity neutralization. Transmembrane action potential signals were digitized and stored on a personal computer for subsequent analysis as described previously (54).

### Immunohistochemistry

Tissue blocks were snap-frozen in liquid nitrogen, and 5 μm serial sections were cut with a cryostat (Microm HM505E) and air dried. Sections were washed in PBS, blocked for 20 minutes with 10% goat serum, and incubated overnight at 4° C. with anti-SkM1 antibody (1:200, Sigma-Aldrich, St Louis, Mo.) and anti-HCN2 antibody (1:200, Alomone, Jerusalem, Israel). Antibody bound to target antigen was detected by incubating sections for 2 hours with goat anti-mouse IgG labeled with Cy3 (red fluorescence for SkM1) and goat anti-rabbit IgG labeled with Alexa 488 (green fluorescence for HCN2), images were collected with a Nikon E800 fluorescence microscope.

### Statistical Analysis

Data are presented as mean±SEM. Two-way repeated measures ANOVA was used to evaluate the effect of an implanted construct on electrophysiological parameters and to test the effect of epinephrine. Subsequent analysis was performed with Bonferroni's test. P<0.05 was considered significant.

### Results

### Intact Animal Studies

In HCN2/GFP injected animals, more then 30% of all beats were triggered by the electronic pacemaker on day 5-8. During these days HCN2/SkM1 injected animals were completely independent from electronic pacing (FIG. 11A; P<0.05). Twenty four hour recordings indeed confirmed presence of biological pacemaker rhythms in more the 95% of the beats. Escape rates (recorded with the electronic pacemaker switched off) in HCN2/SKM1 animals were in the range of 70-80 bpm and significantly faster then rates in HCN2/GFP animals (FIG. 11B; P<0.05). In line with the faster beating rates, escape times after 30 seconds of overdrive suppression, were also significantly shorter in HCN2/SkM1 animals (FIG. 11C; P<0.05).

Upon intravenous administration of epinephrine (1.0 μg/kg per minute), rates significantly increased, from 54±5 to 85±9 bpm in HCN2/GFP animals, and from 75±13 to 117±3 in HCN2/SkM1 animals (P<0.05 vs baseline and HCN2/GFP).

### Tissue Bath and Immunohistochemistry Studies

To confirm contribution of both HCN2 and SkM1 currents to the automaticity seen in HCN2/SkM1 injected animals we performed tissue bath experiments. FIG. 12 demonstrates a typical example of such an experiment in which we superfused the endocardial tissue slab (composed of the injection site, including purkinje fiber and surrounding endocardium) with isoproterenol and TTX, after the TTX washout, with Ivabradine. The reductions in rate seen after the application of TTX and Ivabradine indicate contribution of respectively SkM1 and HCN2 to the isoproterenol enhanced rhythms. Presence of HCN2 and SkM1 proteins was subsequently confirmed using immunohistochemistry (FIG. 13).

### Discussion

The combination of the pacemaker gene HCN2 and the skeletal muscle sodium channel, SkM1, has provided what is thus far the most potentially clinically applicable of any of the biological pacemaker strategies studied so far. For the first time biological pacemaker rhythms are generated in more then 95% of the time, in a large animal model comparable to patients requiring a ventricular on demand pacemaker. Baseline beating rates are within the target range of 70-80 bpm, and demonstrate a brisk autonomic response.

### Example 6

Introducing Long-term HCN2/SkM1-based Biological Pacemaker Function using Lentiviral Gene Transfer

### Vector Construction

The human HCN2 and SkM1 (or mutant/isoform variants) genes are packaged in a single or two separate viral vectors. Third generation lentiviral vectors (as used previously by us)³⁹ are preferably used to introduce a selection of the following modifications to enhance safety and/or improve the efficiency of production; a cardio specific, an inducible or a constitutive promoter (e.g. cardiac tropinin T promoter⁵⁵, or the myosin light chain 2v promoter fused to a minimal CMV enhancer^{56,57}, or Doxycycline sensitive promoter^{58,59}, or a CMV promoter), elements to block leaky gene expression from the viral LTR (e.g. a reversely orientated expression cassette—in combination with a cardiac specific promoter, or the introduction of insulators—such as a core element of the chicken β-globin insulator⁶⁰), elements to block expression in antigen presenting cells (e.g., using post-transcriptional control by endogenous micro RNAs; e.g. microRNA-155 target sites⁶¹), and/or elements to further enhance pacemaker function (e.g. nucleic acids to modify pacemaker related genes such as: Cx genes, AC genes, PDE genes, potassium channel genes, beta subunits, and transcription factors).

### Vector Production & Dose

Lentiviral vectors are initially produced using standard Ca²⁺-phosphate-based transfection (discussed in the methods section) of HEK293T producer cells, to obtain a final concentration of at least 1*10⁹ transducing units (TU)/ml. Vectors are pelleted at 20,000 rpm for two hrs or alternatively at 4,000 rpm for at least 10 hrs, this process is repeated for up two 3 times to obtain the required vector concentration. Special medium formulations are used to increase total vector yield (e.g. addition of caffeine⁶²). In a later stage, to improve the ease of scaling up the production process, stably modified producer cell lines will be used and production will be in GMP-compliant facilities.

To obtain stable biological pacemaker function, it is expected that a dose of 100 to 10,000 μl of total vector mix with both vector (if 2 are used) at, at least 1*10⁹ TU/ml, should suffice. Titers are determined with qPCR and/or immunocytochemistry based methods on serially transduced HEK293T or HeLa cells.

### Vector Testing

Functionality of HCN2/SkM1-based lentiviral vectors are tested in various systems. Efficiency of gene transfer is determined after delivery of a single dose into the left ventricular wall of conventional mice or rats. Long-term expression of human HCN2 and SkM1 genes is studied after gene transfer in immunocompromised mice or rats (e.g. SCID mice, RAG-2/gamma(c)KO mice, or nude rats). Functionality of lentiviral HCN2/SkM1 expression is tested in single NRVMs (using patch-clamp), in NRVMs monolayers (preferably dual population monolayers as described in this application), and in rats (conventional or immunocompromised) after application to the left atrium or left ventricle. Functionality in the rat is studied after inducing significant heart rate slowing using acetylcholine (or analogues) or i.v. adenosine. Finally, when sufficient function is demonstrated in vitro and in small animals, we proceed our testing in canine. Here initial testing is in AV-blocked dogs, with an electronic pacemaker in the right ventricular apex and vector constructs being implanted into the left bundle branch (similar to example 5). At a later stage we will also test functionality of HCN2/SkM1lentiviral vectors after injection into the left and/or right atrium, in SAN/AV-blocked dogs with a dual chamber (atrially sensing and ventricular pacing) electronic pacemaker. In these canine studies, pacemaker function is continuously monitored using pacemaker-log-recordings, 24 hr holter recordings, and daily overdrive suppression. In terminal experiments transduced tissues are harvested and studied in the tissue bath using micro electrodes or optical mapping (using voltage sensitive or Ca²⁺ sensitive dyes). Tissues are also studied using immunohistochemistry, immnocytochemistry, qPCR, Western blotting and gene chip. Gene expression in other organs, such as long and liver, are also studied with qPCR and Western blotting.

### BRIEF DESCRIPTION OF THE DRAWINGS

FIG. 1. Schematic diagram of a non-limiting example of biopacemaker impulse formation combined with impulse protection.

FIG. 2. Improved impulse formation by PDE4 inhibition. A, Neonatal rat cardiac myocytes overexpressing HCN4 demonstrate a shift in the I_{f} activation curve with PDE4 inhibition by 100 nM rolipram. B, Increased spontaneous activity in control neonatal rat cardiac myocytes exposed to PDE4 inhibition by 100 nM rolipram.

FIG. 3. Cardiac progenitor cells transduced with lentiviral vectors. A, FACS analyses of control (upper panel) and LV-GFP transduced (lower panel) CMPCs. A transduction efficiency of nearly 70% was obtained with a multiplicity of infection of 100. B, Typical I_{f} trace of a LV-HCN4-GFP transduced CPC, 2 months after transduction. Fluorescence microscopy of LV-GFP transduced CMPCs (C), LV-HCN4-GFP transduced CMPCs (D) and LV-TBX3-GFP transduced CMPCs (E). F, Increased beating rates in NRCM monolayers co-cultured with HCN4 transduced CMPCs as compared to NRCM monolayers co-cultured with GFP transduced CMPCs. G, Optical activation map of spontaneous biopacemaker activity in a monolayer of a LV-HCN4-CMPC/NRCM hybrid monolayer. Optical action potentials of the indicated detectors are shown below the activation maps, arrows indicate phase 4 diastolic depolarization.

FIG. 4. Improved central activation by partial uncoupling. Typical examples activation maps of monolayers demonstrating peripheral (A), central (B) and re-entry (C) activation. D, Combined data are summarized in the Table of this figure. Immunofluorescence demonstrates overexpression of both HCN4 (green) and Cx43Δ (red) in the central area (E). The peripheral area of the monlayer demonstrates normal Cx43 expression and lack of HCN4 expression (F). Nuclei are counter stained using DAPI.

FIG. 5. Inducible TBX3 overexpression in adult myocardium. Quantitative PCR analysis on samples of 6 left atria of 3 tamoxifen-treated MCM-CT3 mice and 3 tamoxifen-treated MCM mice as controls. Connexin 43 and 40 were down regulated by 17% and 13% (p-value<0.01), respectively.

FIG. 6. In vitro characterization of lentiviral TBX3 overexpression. A, Pulse protocol, Net current was measured at the beginning and the end of hyper- and depolarizing voltage clamp step (panel B) and by stepping back to the holding potential of −40 mV (panel C). B, Average I-V relationship of net membrane currents at begin (I_{begin}) and end (I_{end}) of the voltage steps.TBX3 overexpressing cells as well as controls do not demonstrate a larger I_{end} compared to I_{begin}, indicative for absence of a large I_{f}. In TBX3 overexpressing cells, we observed a reduced instantaneous current in the voltage range of I_{Ca,L} activation and a reduced steady-state current in the voltage range of I_{K1} activation. C, Absence of current activation in TBX3 overexpressing cells after voltage clamp steps demonstrates strongly reduced I_{Na}. D, TBX3 overexpressing cells demonstrate increased firing frequency, less negative MDP and slower upstroke velocity, characteristics of nodal cells.

FIG. 7. In vitro characterization of EVY deleted human HCN4 Immunolabelling of wild type (A) and EVY deleted HCN4 (B). C, Patch-clamp analysis demonstrates significantly slowed activation and deactivation kinetics, together with insignificant changes in the other biophysical properties.

FIG. 8. In vitro screening system to investigate various solutions to current-to-load mismatch based biopacemaker instabilities. Key in this system is the surrounding of biopacemaker cells by normal myocytes. This configuration can be obtained via two different methods: A, Virally transduced biopacemaker cells surrounded by non-pacemaker cells can also be obtained via focal transductions of myocyte monolayers. Lentiviral particles are complexed to magnetic nanoparticles and injected above the myocyte monolayer. The complexed viruses are subsequently attracted to the central area using custom-made, strong magnets. B, Patterned seeding of biopacemaker stem cells or transduced cardiac myocytes in a central area. Here, initial seeding is limited to the centre of the coverslip using silicon or polysulfon rings. After initial seeding and attachment, rings are removed and freshly isolated neonatal myocytes are seeded, to cover the full coverslip. C, Schematic drawing of the tandem lens optical mapping setup; see methods-text for details.

FIG. 9. Biopacemaker instabilities in vitro. A, Typical recordings of cycle lengths from monolayers heterogeneously expressing GFP or HCN4 throughout the monolayer (MOI: Multiplicity of Infection). Upper panels represent baseline and lower panels represent measurements 10 min after the addition of DBcAMP on the same monolayer. B, Typical optical activation maps of dual population monolayers having centers of (left-to-right) GFP or HCN4 expressing cells surrounded by non-pacemaker cells. HCN4 monolayers demonstrate central activation and phase-4 depolarization (black arrows), whereas GFP monolayers demonstrate absence of both. C, Optical action potential recordings from two different dual population HCN4 monolayers. Top: Typical recording of cycle length instabilities (dashed arrows; same monolayer as depicted in panel B. Bottom: Typical recording of “on-off-switching”. Gray arrows indicate subthreshold depolarizations, black arrow indicates initiation of temporarily stable central activation.

FIG. 10. In vivo lentiviral gene transfer. A, Injection of LV-GFP into the left ventricle free wall. B, Fluorescence microscopy of in vivo transduced myocytes expressing GFP. C, Three-dimensional reconstruction of the transduced area after a single injection of 50 μl LV-GFP (1*10⁹ TU/ml), the transduced area extends over approximately one third of the rat left ventricle free wall.

FIG. 11. In intact animal experiments. A, In HCN2/SkM1 animals, percentage of electronically stimulated beats was significantly reduced to 0% on day 4-8, demonstrating absence of biopacemaker dysfunction during these days. Note that the percentages of electronically stimulated beats start out identical. B, Escape rates of HCN2/SkM1 animals were significantly faster then animals overexpressing HCN2-GFP. C, Escape rates were recorded after 30 seconds of overdrive suppression and were significantly shorter in HCN2/SkM1 animals; * indicates P<0.05.

FIG. 12. In tissue bath experiments, LBB preparations of HCN2-SkM1 injected animals demonstrate robust spontaneous activity sensitive to isoproterenol (present throughout the protocol after initial application), to TTX (demonstrating a critical role for SkM1 in the rate enhancements; 0.1 μM TTX specifically blocks SkM1 and did not have a significant effect on spontaneous activity in HCN2/GFP preparation), and after TTX washout, to Ivabradine, indicating contribution of HCN2 to the isoproterenol stimulated rhythm.

FIG. 13. Immunohistochemistry in a HCN2/SkM1 injected animal. A, injected region is positive for HCN2 (green) and SkM1(red). Nuclei are counter stained using DAPI. B, non-injected region is negative for HCN2 and SkM1.

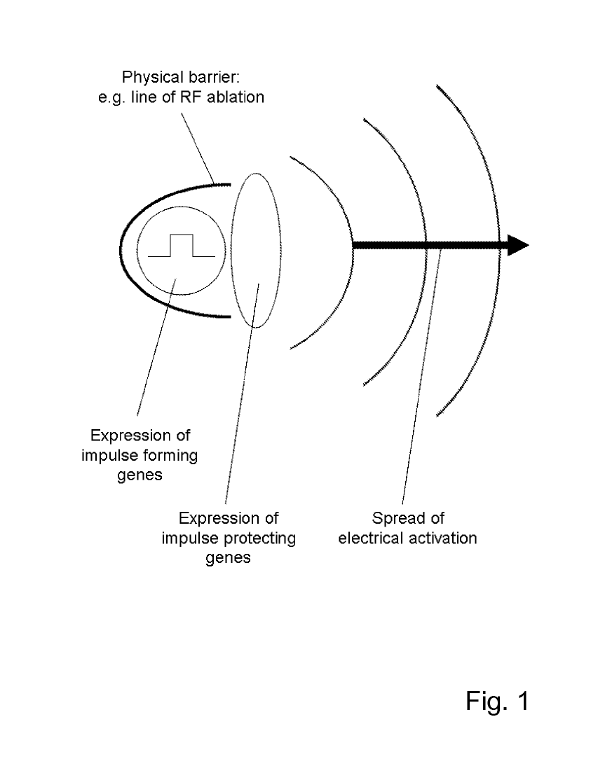

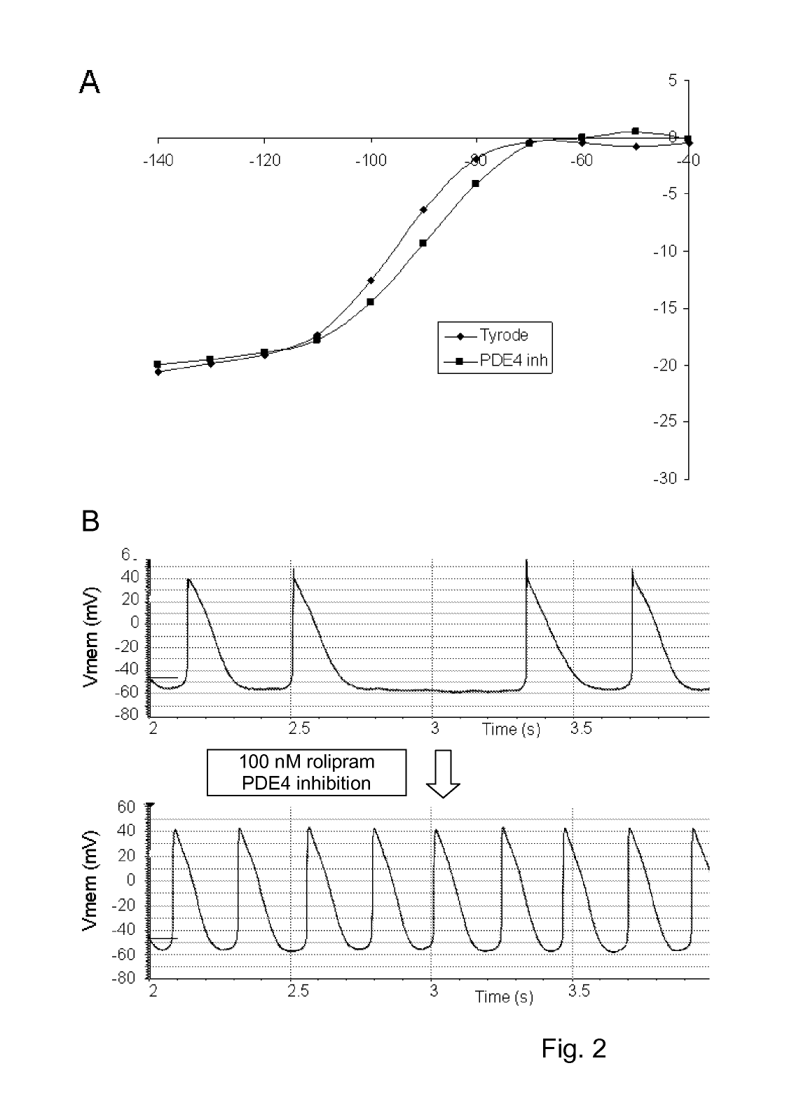

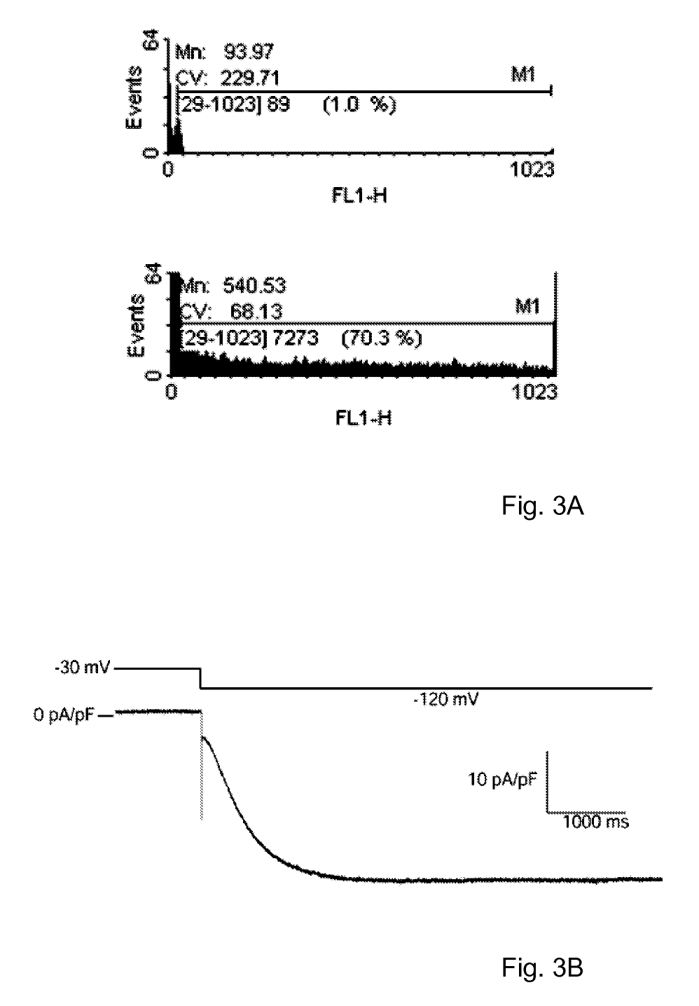

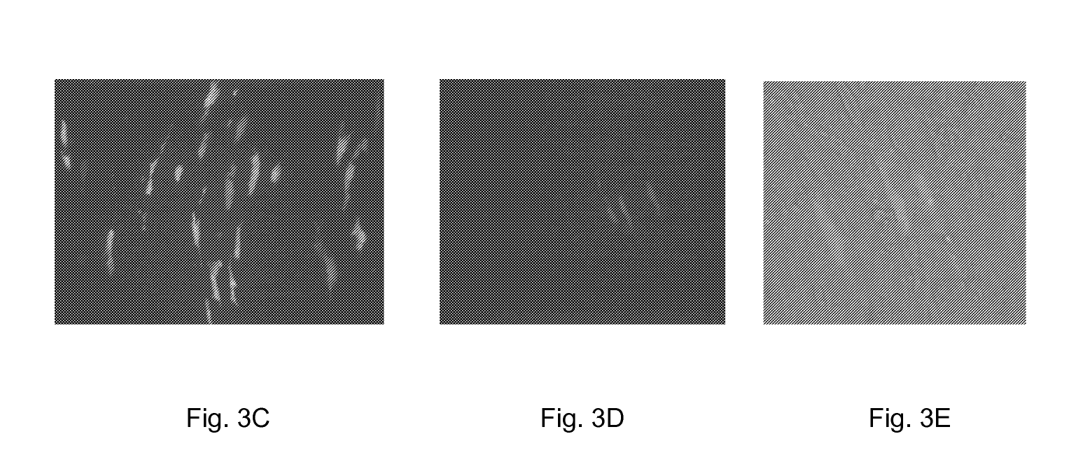

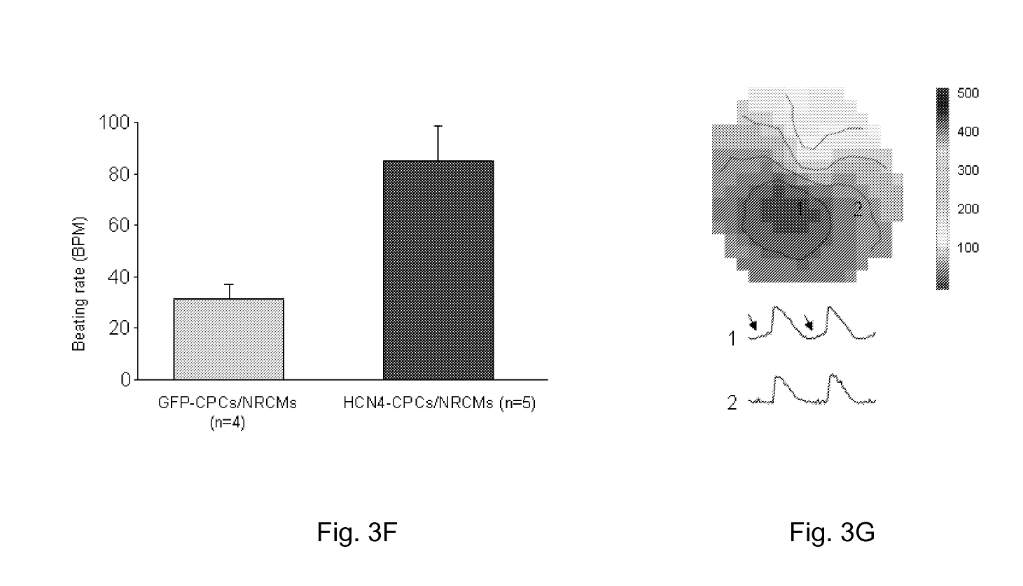

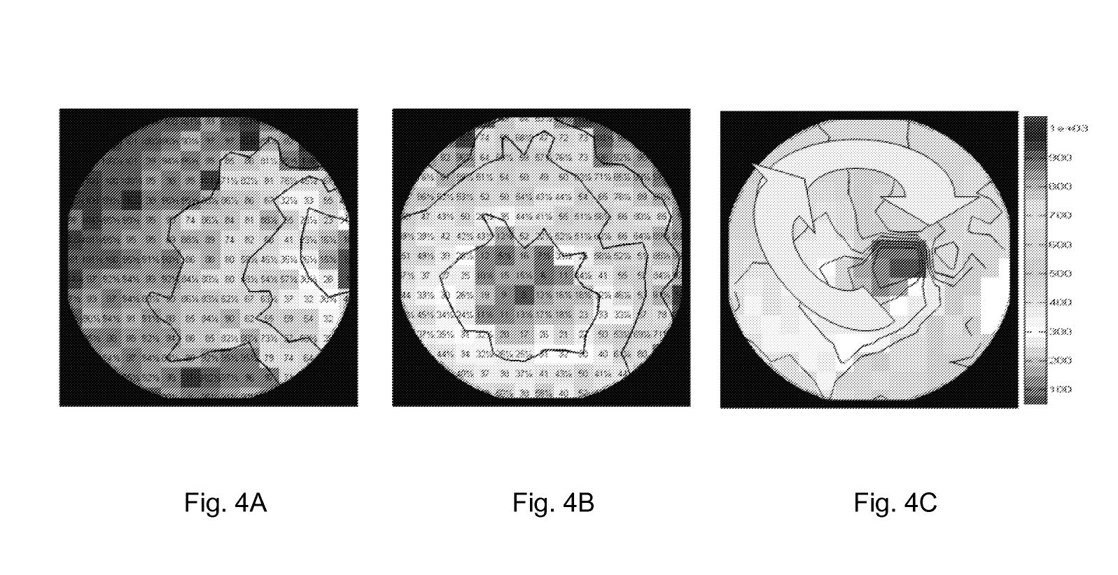

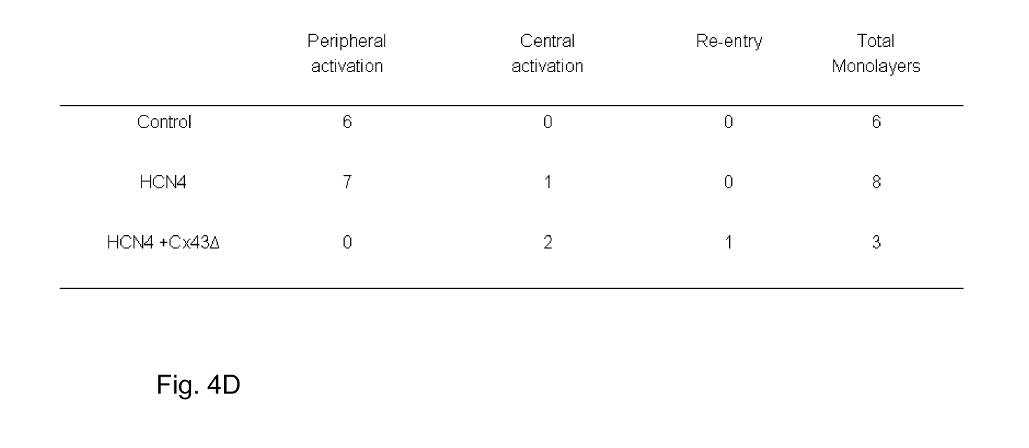

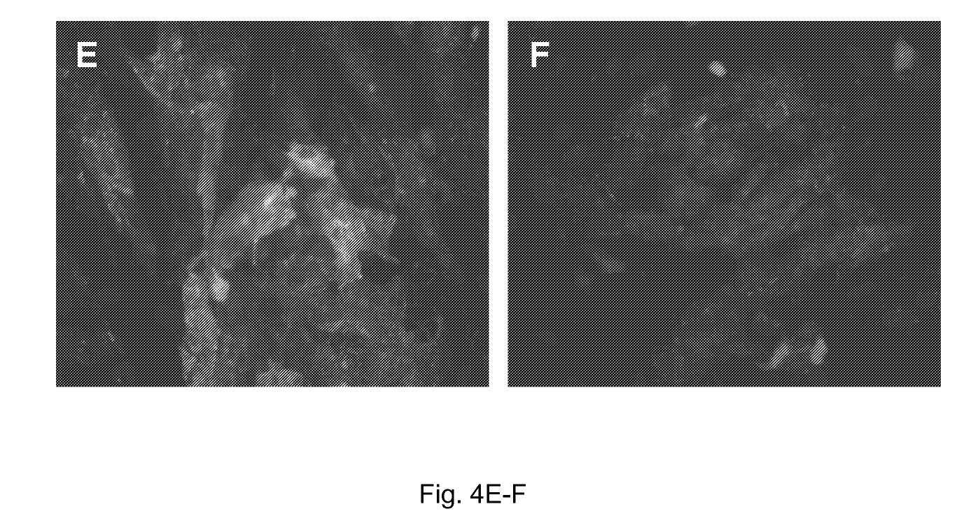

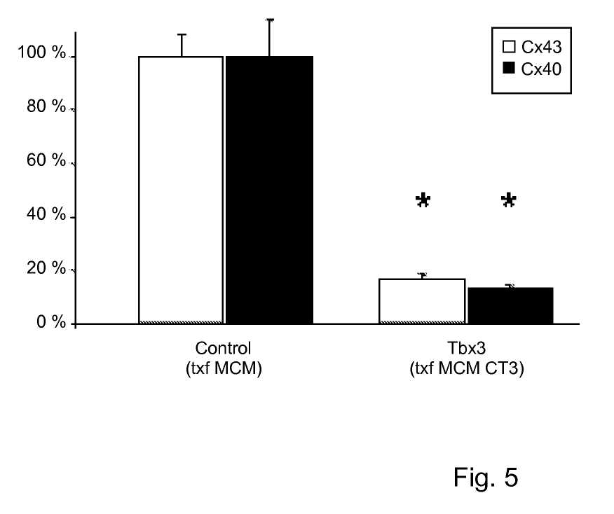

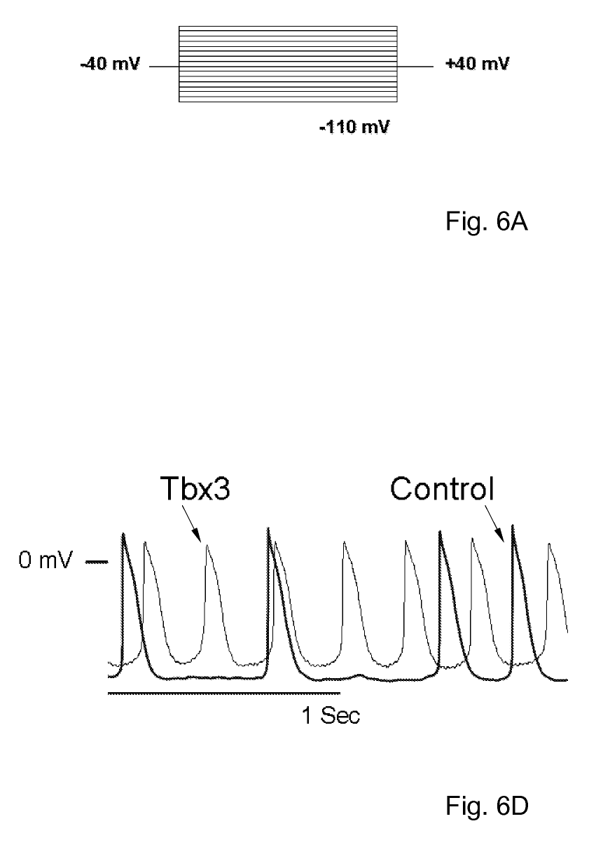

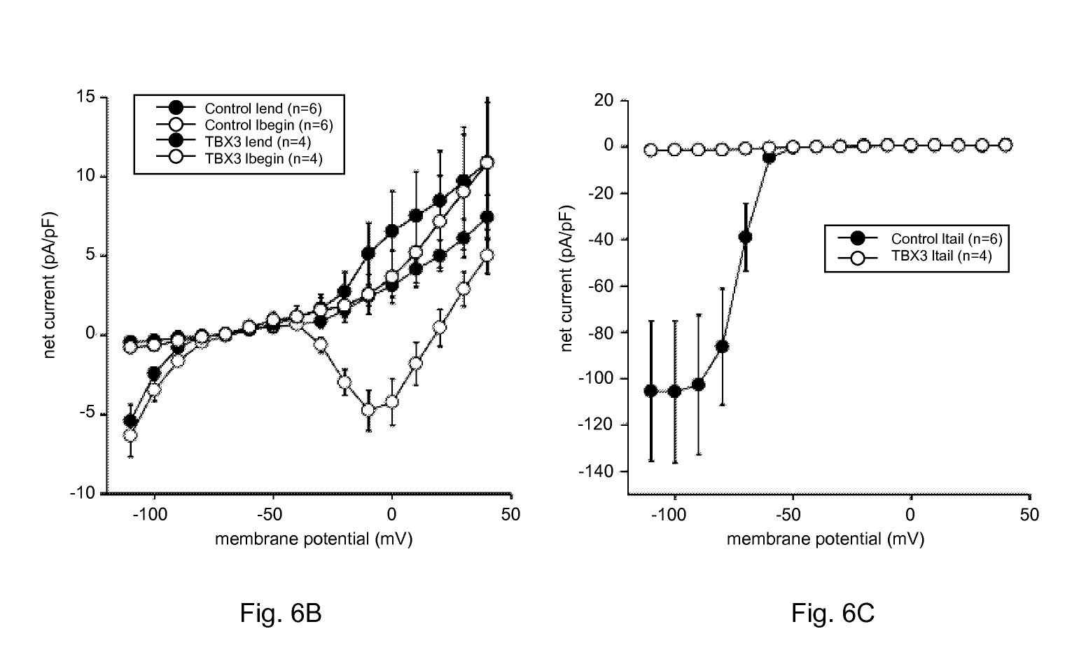

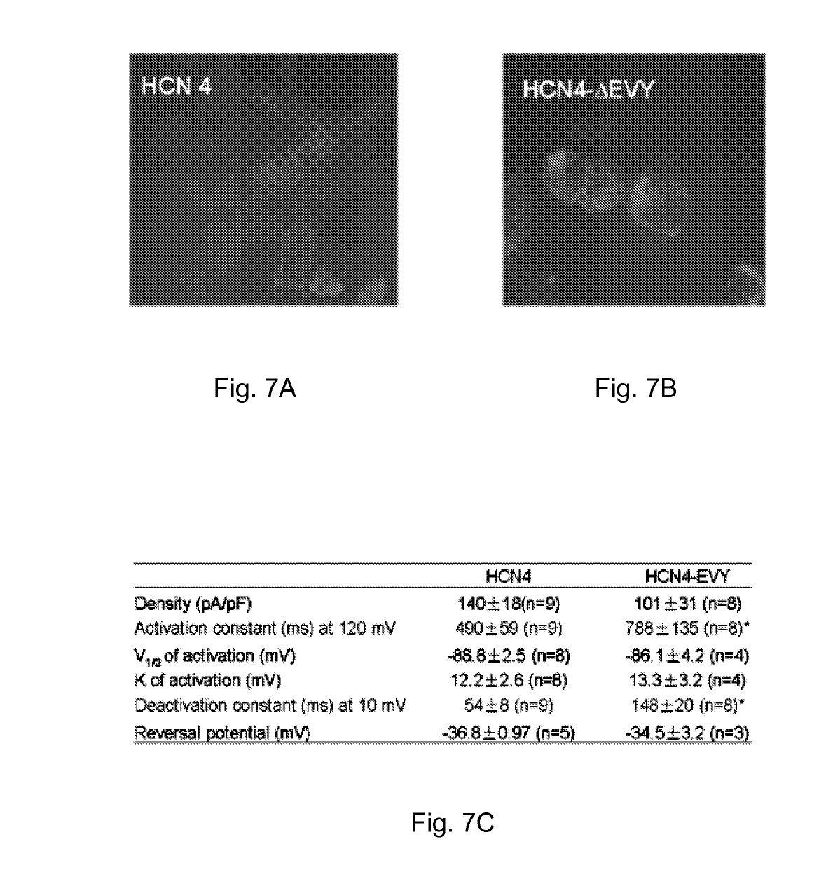

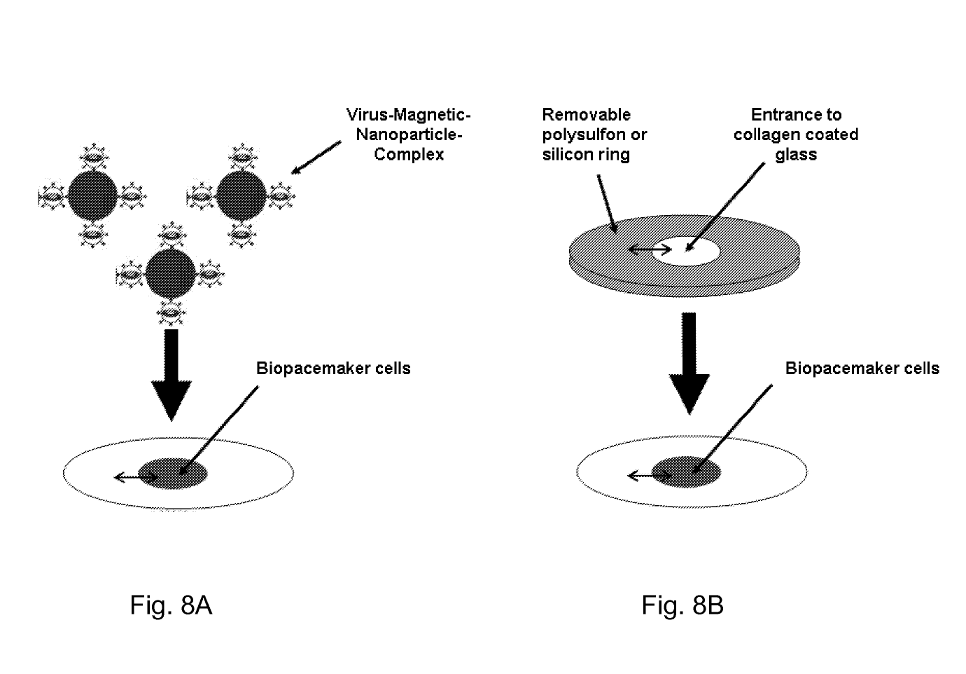

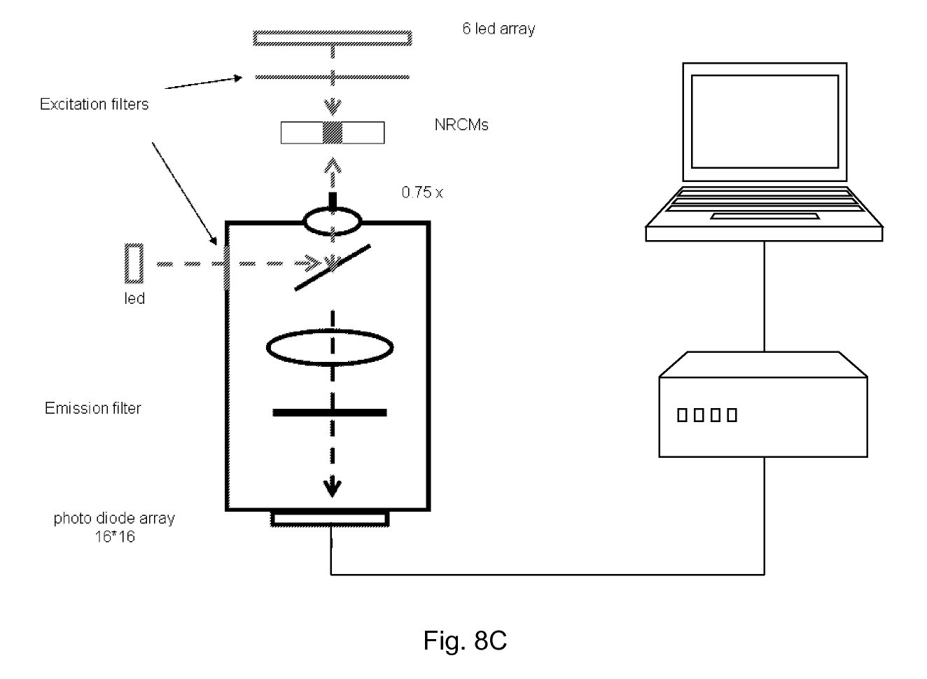

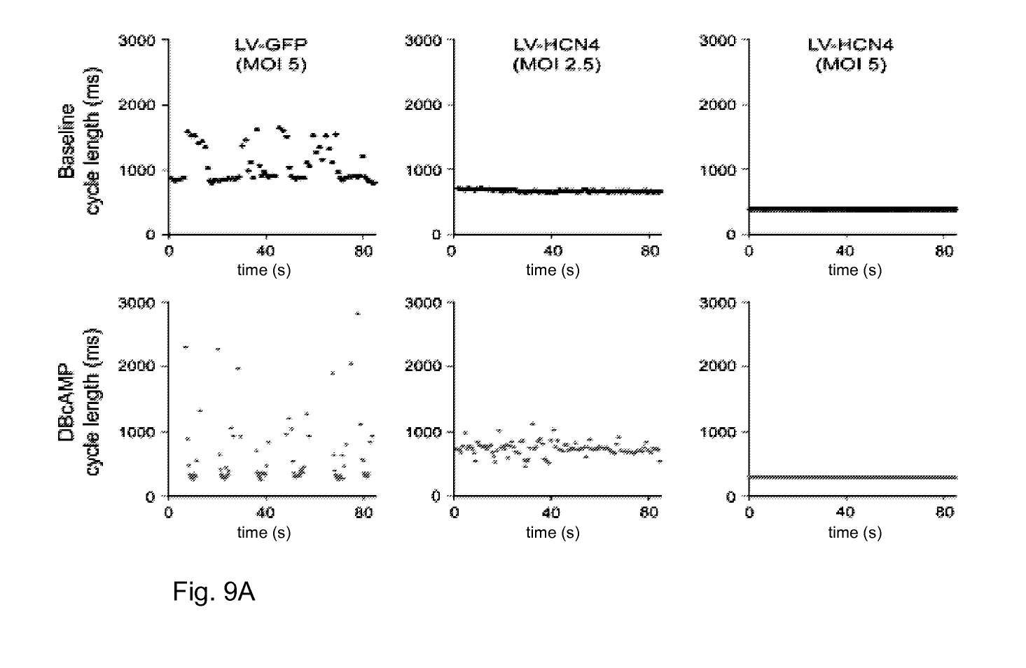

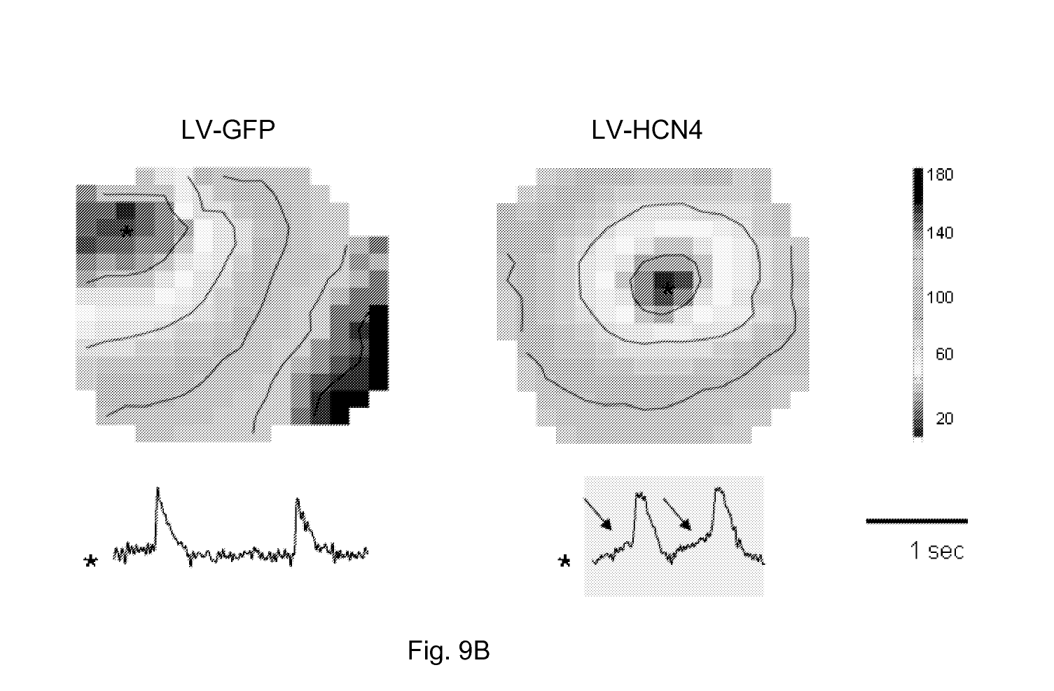

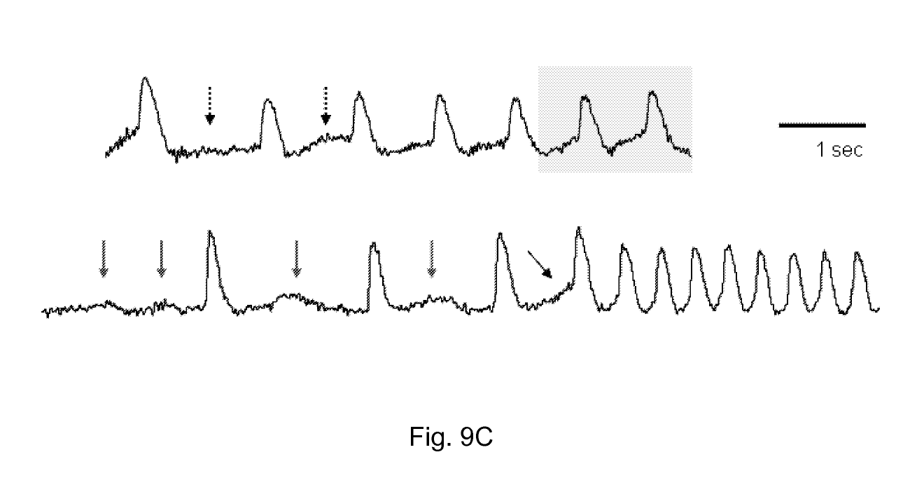

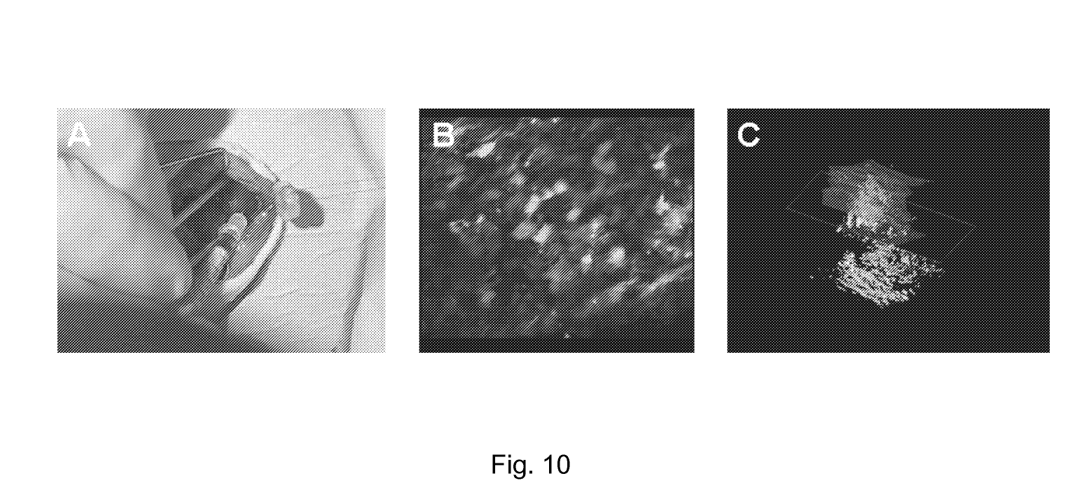

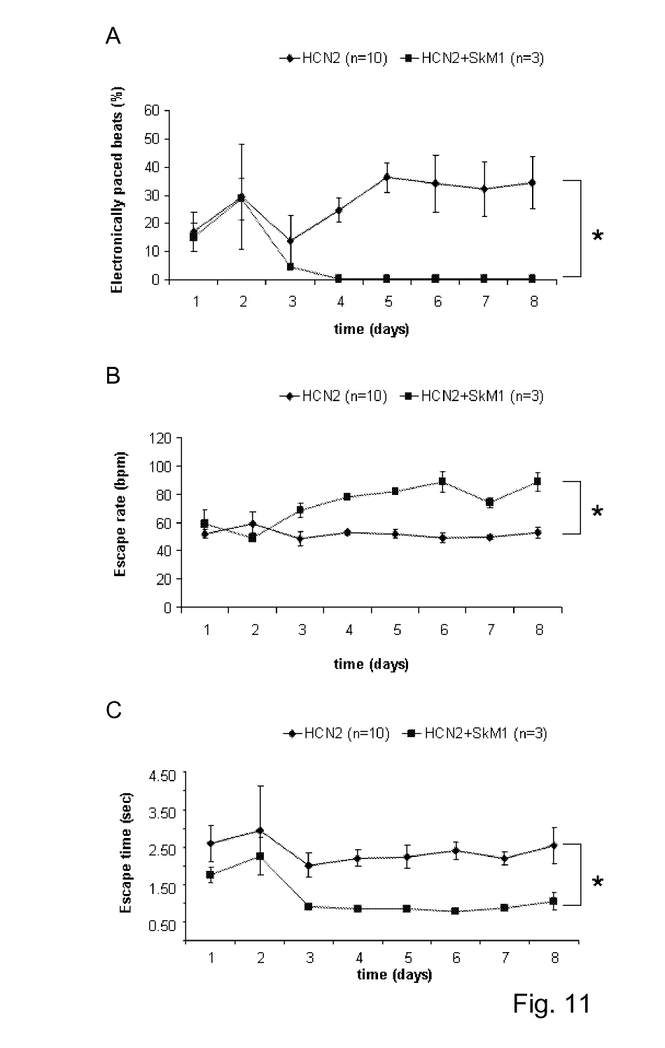

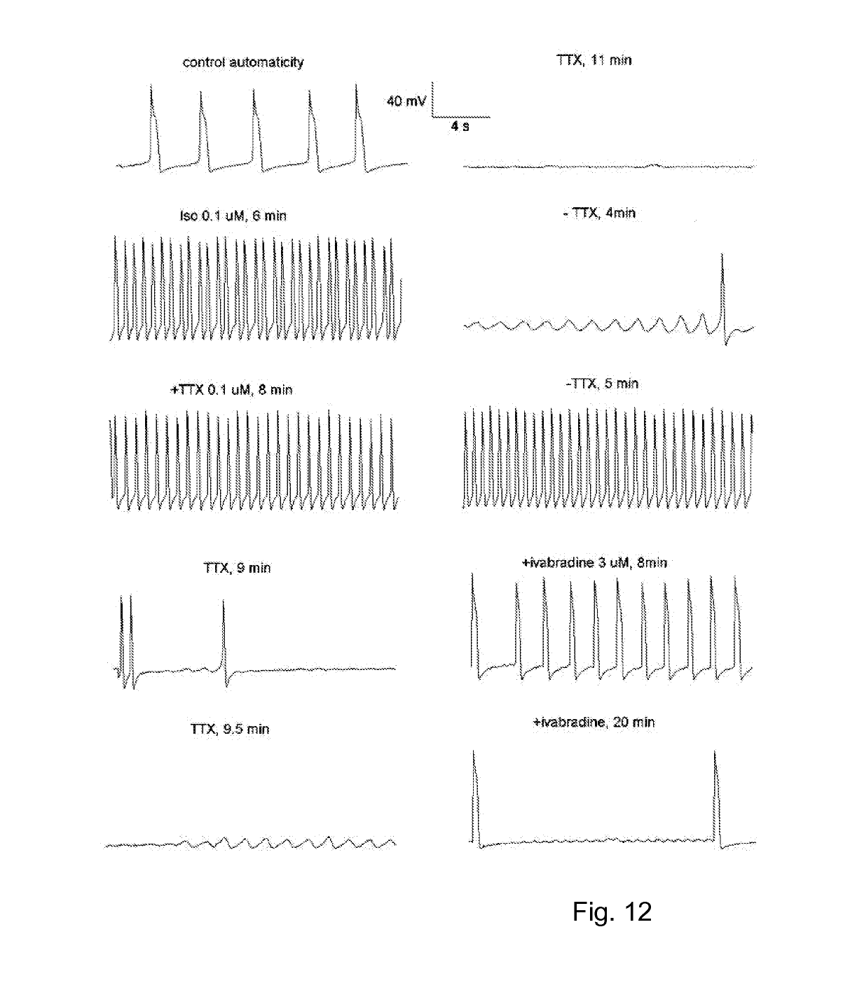

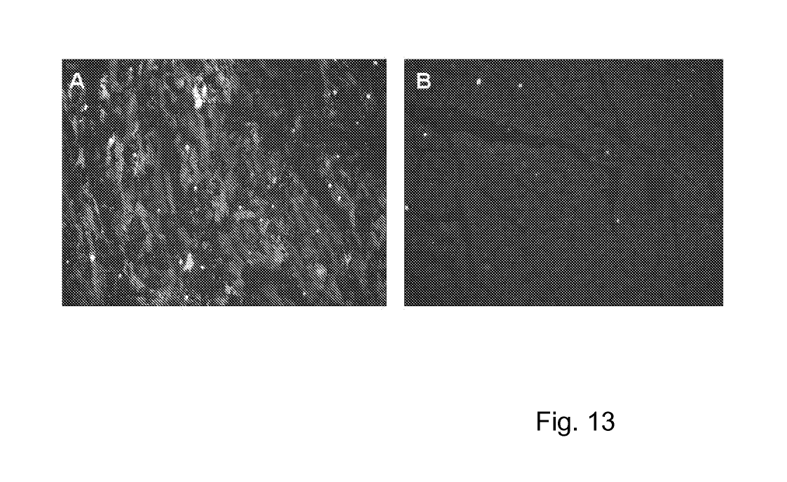

### REFERENCES

1. Qu J, Plotnikov A N, Danilo P, Jr., Shlapakova I, Cohen I S, Robinson RB et al. Expression and function of a biological pacemaker in canine heart. Circulation 2003: 107(8):1106-1109.

2. Miake J, Marbán E, Nuss H B. Biological pacemaker created by gene transfer. Nature 2002: 419(6903):132-133.

3. Edelberg J M, Huang D T, Josephson M E, Rosenberg R D. Molecular enhancement of porcine cardiac chronotropy. Heart 2001: 86(5):559-562.

4. Potapova I, Plotnikov A, Lu Z, Danilo P, Jr., Valiunas V, Qu J et al. Human mesenchymal stem cells as a gene delivery system to create cardiac pacemakers. Circ Res 2004: 94(7):952-959.

5. Plotnikov A N, Sosunov E A, Qu J, Shlapakova I N, Anyukhovsky E P, Liu L et al. Biological pacemaker implanted in canine left bundle branch provides ventricular escape rhythms that have physiologically acceptable rates. Circulation 2004: 109(4):506-512.

6. Kehat I, Khimovich L, Caspi O, Gepstein A, Shofti R, Arbel G et al. Electromechanical integration of cardiomyocytes derived from human embryonic stem cells. Nat Biotechnol 2004: 22(10):1282-1289.

7. Plotnikov A N, Shlapakova I N, Kryukova Y, Bucchi A, Pan Z M, Danilo P J et al. Comparison of mHCN2 and mHCN2-E324A genes as biological pacemakers. Circulation 2005: 112(17):U180.

8. Xue T, Cho H C, Akar F G, Tsang S Y, Jones S P, Marban E et al. Functional integration of electrically active cardiac derivatives from genetically engineered human embryonic stem cells with quiescent recipient ventricular cardiomyocytes: insights into the development of cell-based pacemakers. Circulation 2005: 111(1):11-20.

9. Bucchi A, Plotnikov A N, Shlapakova I, Danilo P, Jr., Kryukova Y, Qu J et al. Wild-type and mutant HCN channels in a tandem biological-electronic cardiac pacemaker. Circulation 2006: 114(10):992-999.

10. Tse H F, Xue T, Lau C P, Siu CW, Wang K, Zhang Q Y et al. Bioartificial sinus node constructed via in vivo gene transfer of an engineered pacemaker HCN Channel reduces the dependence on electronic pacemaker in a sick-sinus syndrome model. Circulation 2006: 114(10):1000-1011.

11. Kashiwakura Y, Cho H C, Barth A S, Azene E, Marban E. Gene transfer of a synthetic pacemaker channel into the heart: a novel strategy for biological pacing. Circulation 2006: 114(16):1682-1686.

12. Cho H C, Kashiwakura Y, Marban E. Creation of a biological pacemaker by cell fusion. Circ Res 2007: 100(8):1112-1115.

13. Plotnikov A N, Shlapakova I, Szabolcs M J, Danilo P, Jr., Lorell B H, Potapova I A et al. Xenografted Adult Human Mesenchymal Stem Cells Provide a Platform for Sustained Biological Pacemaker Function in Canine Heart. Circulation 2007.

14. Qu J, Barbuti A, Protas L, Santoro B, Cohen I S, Robinson R B. HCN2 overexpression in newborn and adult ventricular myocytes: distinct effects on gating and excitability. Circ Res 2001: 89(1):E8-14.

15. Qu J, Kryukova Y, Potapova I A, Doronin S V, Larsen M, Krishnamurthy G et al. MiRP1 modulates HCN2 channel expression and gating in cardiac myocytes. J Biol Chem 2004: 279(42):43497-43502.

16. Vinogradova T M, Lyashkov A E, Zhu W, Ruknudin A M, Sirenko S, Yang D et al. High basal protein kinase A-dependent phosphorylation drives rhythmic internal Ca2+ store oscillations and spontaneous beating of cardiac pacemaker cells. Circ Res 2006: 98(4):505-514.

17. Mattick P, Parrington J, Odia E, Simpson A, Collins T, Terrar D. Ca2+-stimulated adenylyl cyclase isoform AC1 is preferentially expressed in guinea-pig sino-atrial node cells and modulates the I(f) pacemaker current. J Physiol 2007: 582(Pt 3):1195-1203.

18. Rose R A, Kabir M G, Backx P H. Altered Heart Rate and Sinoatrial Node Function in Mice Lacking the cAMP Regulator Phosphoinositide 3-Kinase-{gamma} Circ Res 2007.

19. Kerfant B G, Rose R A, Sun H, Backx P H. Phosphoinositide 3-kinase gamma regulates cardiac contractility by locally controlling cyclic adenosine monophosphate levels. Trends Cardiovasc Med 2006: 16(7):250-256.

20. Mongillo M, McSorley T, Evellin S, Sood A, Lissandron V, Terrin A et al. Fluorescence resonance energy transfer-based analysis of cAMP dynamics in live neonatal rat cardiac myocytes reveals distinct functions of compartmentalized phosphodiesterases. Circ Res 2004: 95(1):67-75.

21. Joyner R W, van Capelle F J. Propagation through electrically coupled cells. How a small S A node drives a large atrium. Biophys J 1986: 50(6):1157-1164.

22. Joyner R W, Wilders R, Wagner M B. Propagation of pacemaker activity. Med Biol Eng Comput 2006.

23. Rohr S, Kucera J P, Fast V G, Kleber A G. Paradoxical improvement of impulse conduction in cardiac tissue by partial cellular uncoupling. Science 1997: 275(5301):841-844.

24. Seppen J, Rijnberg M, Cooreman M P, Oude Elferink R P J. Lentiviral vectors for efficient transduction of isolated primary quiescent hepatocytes. J Hepatol 2002: 36(4):459-465.

25. Seppen J, van der Rijt R, Looije N, van Til N P, Lamers W H, Oude Elferink R P J. Long-term correction of bilirubin UDPglucuronyltransferase deficiency in rats by in utero lentiviral gene transfer. Mol Ther 2003: 8(4):593-599.

26. Rohr S, Schöllly D M, Kléber A G. Patterned growth of neonatal rat heart cells in culture. Morphological and electrophysiological characterization. Circ Res 1991: 68(1):114-130.

27. Ludwig A, Zong X, Jeglitsch M, Hofmann F, Biel M. A family of hyperpolarization-activated mammalian cation channels. Nature 1998: 393(6685):587-591.

28. Er F, Larbig R, Ludwig A, Biel M, Hofmann F, Beuckelmann D J et al. Dominant-negative suppression of HCN channels markedly reduces the native pacemaker current I(f) and undermines spontaneous beating of neonatal cardiomyocytes. Circulation 2003: 107(3):485-489.

29. van Ginneken A C, Giles W. Voltage clamp measurements of the hyperpolarization-activated inward current I(f) in single cells from rabbit sino-atrial node. J Physiol 1991: 434:57-83.

30. Potse M, Linnenbank A C, Grimbergen C A. Software design for analysis of multichannel intracardial and body surface electrocardiograms. Comput Methods Programs Biomed 2002: 69(3):225-236.

31. Hoogaars W M, Engel A, Brons J F, Verkerk A O, de Lange F J, Wong LY et al. TBX3 controls the sinoatrial node gene program and imposes pacemaker function on the atria. Genes Dev 2007: 21(9):1098-1112.

32. Brummelkamp T R, Kortlever R M, Lingbeek M, Trettel F, MacDonald M E, van L M et al. TBX-3, the gene mutated in Ulnar-Mammary Syndrome, is a negative regulator of p19ARF and inhibits senescence. J Biol Chem 2002: 277(8):6567-6572.

33. Sohal D S, Nghiem M, Crackower M A, Witt S A, Kimball T R, Tymitz K M et al. Temporally regulated and tissue-specific gene manipulations in the adult and embryonic heart using a tamoxifen-inducible Cre protein. Circ Res 2001: 89(1):20-25.

34. Novak A, Guo C, Yang W, Nagy A, Lobe C G. Z/EG, a double reporter mouse line that expresses enhanced green fluorescent protein upon Cre-mediated excision. Genesis 2000: 28(3-4):147-155.

35. Karlen Y, McNair A, Perseguers S, Mazza C, Mermod N. Statistical significance of quantitative PCR. BMC Bioinformatics 2007: 8:131.

36. Bucchi A, Plotnikov A N, Shlapakova I, et al. Wild-type and mutant HCN channels in a tandem biological-electronic cardiac pacemaker. Circulation 2006: 114:992-999.

37. Cai J, Yi F F, Li Y H, et al. Adenoviral gene transfer of HCN4 creates a genetic pacemaker in pigs with complete atrioventricular block. Life Sci 2007: 80:1746-1753.

38. Plotnikov A N, Bucchi A, Shlapakova I, Danilo P Jr, Brink P R, Robinson R B, Cohen I S, Rosen M R. HCN212-channel biological pacemakers manifesting ventricular tachyarrhythmias are responsive to treatment with I(f) blockade. Heart Rhytm 2008: 5: 282-8.

39. Boink G J J, Verkerk A O, van Amersfoorth S C M, Tasseron S J, van der Rijt R, Bakker D, Linnenbank A C, van der Meulen J, de Bakker J M T, Seppen J, Tan H L. Engineering physiologically controlled pacemaker cells with lentiviral HCN4 gene transfer. J Gene Med. 2008;10;487-97.

40. Boink G J J, Seppen J, de Bakker J M T, Tan H L. Biological pacing by gene and cell therapy. Neth Heart J. 2007;15;318-22.

41. Boink G J J, Seppen J, de Bakker J M T, Tan H L. Gene therapy to create biological pacemakers. Med Biol Eng Comput. 2007;45;167-76.

42. Boink G J J, Bakker M L, Verkerk A O, Bakker D, van Amersfoorth S C M, de Bakker J M T, Seppen J, Christoffels V M, Tan H L. TBX3 as potential target in biopacemaker gene and cell therapy. Hum Gene Ther. 2008 (meeting abstract).

43. Boink G J J, Sluijter J P, Verkerk A O, Bakker D, van Amersfoorth S C M, de Boer T P, van der Heyden M A G, van Veen T A B, Goumans M J, Seppen J, de Bakker J M T, Tan H L. Constructing a biological pacemaker from cardiac progenitor stem cells. Hum Gene Ther. 2008 (meeting abstract).

44. Boink G J J, Bakker M L, Verkerk A O, Bakker D, de Bakker J M T, Seppen J, Christoffels V M, Tan H L. Inducible TBX3 overexpression as a tool for biopacemaker engineering. Circulation. 2008 (meeting abstract).

45. George A L Komisarof J Kallen R G, Barchi R L. Primary structure of the adult human skeletal muscle voltage-dependent sodium channel. Ann. Neurol. 1992: 31: 131-137.

46. Wieland S J, Gong G H, Poblete H, Fletcher J E, Cheni L Q, Kalleni R G. Modulation of Human Muscle Sodium Channels by Intracellular Fatty Acids Is Dependent on the Channel Isoform. J Bio Chem 1996: 271: 19037-041.

47. Lau D H, Clausen C, Sosunov E A, Shlapakova I N, Anyukhovsky E P, Danilo P Jr, Rosen T S, Kelly C, Duffy H S, Szabolcs M J, Chen M, Robinson R B, Lu J, Kumari S, Cohen I S, Rosen M R. Epicardial border zone overexpression of skeletal muscle sodium channel SkM1 normalizes activation, preserves conduction, and suppresses ventricular arrhythmia: an in silico, in vivo, in vitro study. Circulation 2009: 6; 19-27.

48. Protas L, Dun W, Jia Z, Lu J, Bucchi A, Kumari S, Chen M, Cohen I S, Rosen M R, Entcheva E, Robinson R B. Expression of skeletal but not cardiac Na+channel isoform preserves normal conduction in a depolarized cardiac syncytium. Cardiovasc Res. 2009: 81:528-35.

49. Bowlby M R, Levitan I B. Block of Cloned Voltage-Gated Potassium Channels by the Second Messenger Diacylglycerol Independent of Protein Kinase C. Journal of Neurophysiology 1995: 73: 2221-9.

50. Yankelson L, Feld Y, Bressler-Stramer T, Itzhaki I, Huber I, Gepstein A, Aronson D, Marom S, Gepstein L. Cell therapy for modification of the myocardial electrophysiological substrate. Circulation 2008: 117:720-31.

51. Feld Y, Melamed-Frank M, Kehat I, Tal D, Marom S, Gepstein L. Electrophysiological modulation of cardiomyocytic tissue by transfected fibroblasts expressing potassium channels: a novel strategy to manipulate excitability. Circulation 2002: 105: 522-9.

52. Ma H, Sumbilla C M, Farrance I K G, Klein M G, Inesi G. Cell-specific expression of SERCA, the exogenous Ca2+ transport ATPase in cardiac myocytes. Am J Physiol Cell Physiol 2004: 286: C556-C564.

53. Sambrook J, Russell D W, Molecular cloning, a laboratory manual, third edition, 2001 Cold Spring Harbour Laboratory Press, Cold Spring Harbour, N.Y.

54. Anyukhovsky E P, Sosunov E A, Rosen M R. Regional differences in electrophysiological properties of epicardium, midmyocardium, and endocardium. In vitro and in vivo correlations. Circulation 1996; 94(8):1981-1988.

55. Wang G, Yeh H I, Lin J J. Characterization of cis-regulating elements and trans-activating factors of the rat cardiac troponin T gene. J Biol Chem 1994 December 2;269(48):30595-603.

56. Gruh I, Wunderlich S, Winkler M et al. Human CMV immediate-early enhancer: a useful tool to enhance cell-type-specific expression from lentiviral vectors. J Gene Med 2008 January; 10(1):21-32.

57. Muller O J, Leuchs B, Pleger S T et al. Improved cardiac gene transfer by transcriptional and transductional targeting of adeno-associated viral vectors. Cardiovasc Res 2006 January 28.

58. Markusic D, Seppen J. Doxycycline regulated lentiviral vectors. Methods Mol Biol 2010;614:69-76.

59. Markusic D M, de Waart D R, Seppen J. Separating lentiviral vector injection and induction of gene expression in time, does not prevent an immune response to rtTA in rats. PLoS One 2010;5(4):e9974.

60. Hanawa H, Yamamoto M, Zhao H, Shimada T, Persons D A. Optimized lentiviral vector design improves titer and transgene expression of vectors containing the chicken beta-globin locus HS4 insulator element. Mol Ther 2009 Apri1;17(4):667-74.

61. Brown B D, Venneri M A, Zingale A, Sergi S L, Naldini L. Endogenous microRNA regulation suppresses transgene expression in hematopoietic lineages and enables stable gene transfer. Nat Med 2006 May;12(5):585-91.

62. Ellis B L, Potts P R, Porteus M H. Creating Higher Titer Lentivirus Using Caffeine. Hum Gene Ther 2010 July 14.

1. A method for providing a cell with a spontaneous electrical activity and/or increasing the depolarization rate of a cell having a spontaneous electrical activity, the method comprising providing a cell with a compound capable of providing and/or increasing a pacemaker current I_{f}, and
diminishing electrical coupling between said cell and surrounding cells; and/or
increasing the availability of I_{Na} at depolarized potentials of said cell, preferably by providing said cell with a sodium channel and/or a functional equivalent of a sodium channel and/or a sodium channel with altered kinetics and/or an alpha subunit of a sodium channel and/or a beta-subunit of a sodium channel; and/or
increasing the firing frequency of said cell by increasing intracellular cAMP and/or by decreasing action potential duration.

2. A method according to claim 1, further comprising reducing the inward rectifier current I_{K1} of said cell.

3. A method according to claim 1, wherein said cell is provided with a hyperpolarization-activated cyclic nucleotide-gated (HCN) channel or a functional equivalent thereof.

4. A method according to claim 1, comprising enhancing the basal cAMP level within said cell.

5. A method according to claim 4, wherein said basal cAMP level is enhanced by increasing the amount and/or activity of a cAMP producing enzyme within said cell.

6. A method according to claim 5, wherein said enzyme comprises an adenylate cyclase.

7. A method according to claim 5, wherein said enzyme comprises adenylate cyclase-1 and/or adenylate cyclase-8.

8. A method according to claim 4, wherein said basal cAMP level is enhanced by reducing the amount and/or activity of an enzyme involved with cAMP breakdown.

9. A method according to claim 8, wherein said enzyme comprises a phosphodiesterase.

10. A method according to claim 1, wherein said cell is provided with:
an siRNA and/or an antisense nucleotide sequence against a phosphodiesterase; and/or
a nucleic acid sequence or a functional equivalent thereof encoding a phosphodiesterase with a diminished function as compared to wild type phosphodiesterase.

11. A method according to claim 10, wherein said nucleic acid sequence or functional equivalent thereof encodes a phosphodiesterase with a dominant diminished function as compared to wild type phosphodiesterase

12. (canceled)

13. (canceled)

14. (canceled)

15. (canceled)

16. (canceled)

17. A method according to claim 1, wherein the electrical coupling between said cell and surrounding cells is diminished by reducing the amount and/or activity of gap junction proteins connecting said cell and surrounding cells.

18. A method according to claim 1, wherein the electrical coupling between said cell and surrounding cells is diminished by providing said cell with a gap junction protein with a diminished conductor capacity as compared to connexin 43 or connexin 40.

19. A method according to claim 1, wherein the electrical coupling between said cell and surrounding cells is diminished by reducing the amount and/or activity of connexin 43 and/or connexin 40 of said cell.

20. A method according to claim 1, wherein said cell is provided with:
an siRNA and/or an antisense nucleotide sequence against connexin 43; and/or
an siRNA and/or an antisense nucleotide sequence against connexin 40; and/or
a nucleic acid sequence or a functional equivalent thereof encoding a connexin with a lower conductor capacity than the conductor capacity of connexin 43; and/or
a nucleic acid sequence or a functional equivalent thereof encoding a connexin with a lower conductor capacity than the conductor capacity of connexin 40.

21. A method according to claim 20, wherein said connexin with a lower conductor capacity as compared to the conductor capacity of connexin 43 or connexin 40 comprises connexin 30.2, connexin 45, connexin 43Δ or a functional equivalent thereof.

22. (canceled)

23. (canceled)

24. A method according to claim 1, further comprising providing said cell with a beta-subunit for a voltage gated potassium channel and/or a nucleic acid sequence or a functional equivalent thereof encoding a beta-subunit for a voltage gated potassium channel.

25. (canceled)

26. A method according to claim 1, wherein the inward rectifier current I_{K1} is reduced by providing said cell with
an siRNA and/or an antisense nucleotide sequence against an inwardly-rectifying channel; and/or
a nucleic acid sequence or a functional equivalent thereof encoding an inwardly-rectifying channel with a diminished function as compared to the same kind of inwardly-rectifying channel in a wild type form.

27. A method according to claim 26, wherein said inwardly-rectifying channel comprises a Kir2.1 channel.

28. A method according to claim 1, further comprising providing said cell with a nucleic acid sequence or a functional equivalent thereof encoding an alpha-subunit of a voltage gated sodium channel and/or a beta-subunit of a voltage gated sodium channel.

29. A method according to claim 1, wherein said sodium channel comprises a voltage gated skeletal muscle sodium channel.

30. A method according to claim 29, wherein said voltage gated sodium channel comprises an SkM1channel or SCN4A or a constitutive active variant thereof, preferably the mutant G1306E of SCN4A.

31. A method according to claim 1, wherein said cell is provided with an HCN channel or functional equivalent thereof and with a SkM1channel, or a functional equivalent thereof.

32. A method according to claim 1, wherein said cell is provided with an HCN2 channel or functional equivalent thereof and with a SkM1channel, or a functional equivalent thereof.

33. A method according to claim 1, wherein said cell is present in, or brought into, atrial or ventricular myocardium.

34. A gene delivery vehicle or a vector or an isolated cell comprising:
a nucleic acid sequence or a functional equivalent thereof encoding a hyperpolarization-activated cyclic nucleotide-gated (HCN) channel, and
one or more nucleic acid sequences selected from the group consisting of:
an siRNA and/or antisense nucleotide sequence against a phosphodiesterase, an siRNA and/or antisense nucleotide sequence against connexin 43, an siRNA and/or antisense nucleotide sequence against connexin 40, an siRNA and/or antisense nucleotide sequence against an inwardly-rectifying channel, and a nucleic acid sequence or a functional equivalent thereof encoding a compound selected from the group consisting of:
a cAMP producing enzyme, an adenylate cyclase, adenylate cyclase-1, adenylate cyclase-8, a compound capable of increasing the amount and/or activity of a cAMP producing enzyme, a compound capable of reducing the amount and/or activity of an enzyme involved with cAMP breakdown, a phosphodiesterase with a diminished function as compared to wild type phosphodiesterase, a compound capable of reducing the amount and/or activity of gap junction proteins connecting said cell and surrounding cells, a gap junction protein with a diminished conductor capacity as compared to connexin 43, a gap junction protein with a diminished conductor capacity as compared to connexin 40, a compound capable of reducing the amount and/or activity of connexin 43 of said cell, a compound capable of reducing the amount and/or activity of connexin 40 of said cell, a connexin with a lower conductor capacity than the conductor capacity of connexin 43, a connexin with a lower conductor capacity than the conductor capacity of connexin 40, connexin 30.2 or a functional equivalent thereof, connexin 45 or a functional equivalent thereof, connexin 43 Δ or a functional equivalent thereof, a transcription factor capable of reducing connexin 43 expression, a transcription factor capable of reducing connexin 40 expression, TBX3 or a functional equivalent thereof, an inwardly-rectifying potassium channel with a diminished function as compared to the same kind of inwardly-rectifying potassium channel in a wild type form, and a Kir2.1 channel or a functional equivalent thereof.

35. A method according to claim 1, wherein said cell comprises a myocardial cell.

36. A method according to claim 1, wherein said cell comprises a cardiac stem cell or cardiac progenitor cell.

37. A method for treating a subject suffering from, or at risk of suffering from, a disorder associated with impaired function of a cell with a spontaneous electrical activity, the method comprising:
providing a cell of said subject with spontaneous electrical activity or
increasing the depolarization rate of a cell of said subject or
administering to said subject a therapeutic amount of a gene delivery vehicle and/or a vector and/or a cell according to claim 34.

38. A method for treating a subject suffering from, or at risk of suffering from, a cardiovascular disorder, the method comprising:
providing a myocardial cell of said subject with spontaneous electrical activity or
increasing the depolarization rate of a myocardial cell of said subject or
administering to said subject a therapeutic amount of a gene delivery vehicle or a vector and/or a cell according to claim 34.

39. A method according to claim 38, wherein said gene delivery vehicle and/or vector and/or cell is administered to the atrium or the ventricle of the heart of said subject.

40. A method according to claim 38, wherein said cardiovascular disorder comprises a cardiac conduction disorder, preferably sick sinus syndrome and/or AV nodal block.

41. A method according to claim 37, wherein said cell is provided with an HCN channel, or a functional equivalent thereof, and with a SkM1channel, or a functional equivalent thereof.

42. A device for increasing the depolarization rate of a cell or a group of cells having spontaneous electrical activity, and/or for providing a cell or a group of cells with spontaneous electrical activity, said device comprising:
means for providing a cell with a compound capable of providing and/or increasing a pacemaker current I_{f}, and
means for diminishing electrical coupling between said cell and surrounding cells.

43. A device according to claim 42, wherein said device comprises a catheter.

44. (canceled)

45. A device according to claim 42, wherein said means for providing a cell with a compound capable of providing and/or increasing a pacemaker current I_{f} comprises an element for injection of a nucleic acid sequence.

46. (canceled)

47. A combination of:
a compound capable of providing and/or increasing a pacemaker current I_{f}, and
a compound capable of diminishing electrical coupling between said cell and surrounding cells and/or a compound capable of reducing the inward rectifier current I_{K1} of said cell
for use as a medicament.

48. A method for preventing or contracting a disorder associated with impaired function of a cell with a spontaneous electrical activity, preferably a cardiovascular disorder, the method comprising providing the subject with
a compound capable of providing and/or increasing a pacemaker current I_{f}, and
a compound capable of diminishing electrical coupling between said cell and surrounding cells and/or a compound capable of reducing the inward rectifier current I_{K1} of said cell.

49. A combination er-use according to claim 47, wherein said compound capable of diminishing electrical coupling between said cell and surrounding cells comprises a device comprising means for providing a cell with a compound capable of providing and/or increasing a pacemaker current I_{f}, and means for diminishing electrical coupling between said cell and surrounding cells.

50. A combination according to claim 47, wherein said compound capable of diminishing electrical coupling between said cell and surrounding cells comprises an siRNA and/or antisense nucleotide sequence against connexin 43 and/or an siRNA and/or antisense nucleotide sequence against connexin 40 and/or a nucleic acid sequence encoding a compound selected from the group consisting of
a compound capable of reducing the amount and/or activity of gap junction proteins connecting said cell and surrounding cells, a gap junction protein with a diminished conductor capacity as compared to connexin 43, a gap junction protein with a diminished conductor capacity as compared to connexin 40, a compound capable of reducing the amount and/or activity of connexin 43 of said cell, a compound capable of reducing the amount and/or activity of connexin 40 of said cell, a connexin with a lower conductor capacity than the conductor capacity of connexin 43, a connexin with a lower conductor capacity than the conductor capacity of connexin 40, connexin 30.2 or a functional equivalent thereof, connexin 45 or a functional equivalent thereof, connexin 43Δ or a functional equivalent thereof, a transcription factor capable of reducing connexin 43 expression, a transcription factor capable of reducing connexin 40 expression and TBX3 or a functional equivalent thereof.

51. A combination according to claim 47, wherein said compound capable of providing and/or increasing a pacemaker current I_{f} comprises an siRNA and/or antisense nucleotide sequence against a phosphodiesterase and/or a nucleic acid sequence encoding a compound selected from the group consisting of a cAMP producing enzyme, an adenylate cyclase, adenylate cyclase-1, adenylate cyclase-8, a compound capable of increasing the amount and/or activity of a cAMP producing enzyme, a compound capable of reducing the amount and/or activity of an enzyme involved with cAMP breakdown, and a phosphodiesterase with a diminished function as compared to wild type phosphodiesterase.

52. A combination according to claim 47, wherein said compound capable of reducing the inward rectifier current I_{K1} of said cell comprises an siRNA and/or antisense nucleotide sequence against an inwardly-rectifying potassium channel and/or a nucleic acid sequence encoding a compound selected from the group consisting of an inwardly-rectifying potassium channel with a diminished function as compared to the same kind of inwardly-rectifying potassium channel in a wild type form, and a Kir2.1 channel or a functional equivalent thereof.

53. A pharmaceutical composition, comprising a gene delivery vehicle and/or a vector and/or a cell according to claim 34, and a pharmaceutically acceptable carrier, diluent or excipient.

54. (canceled)

55. A method for producing a system comprising pacemaker cells which are at least in part surrounded by non-pacemaker cells, the method comprising:
providing an area of pacemaker cells produced by a method according to claim 1, said area being bordered by a composition, preferably a ring or cylinder, and
removing the composition and at least in part surrounding the pacemaker area by non-pacemaker cells.

56. A gene delivery vehicle or a vector comprising a cardiac specific promoter and at least one nucleic acid sequence selected from the group consisting of
at least one nucleic acid encoding a compound capable of providing and/or increasing a pacemaker current I_{f}, and
at least one nucleic acid encoding a compound capable of diminishing electrical coupling between a cell and surrounding cells, and
at least one nucleic acid encoding a compound capable of increasing the availability of I_{Na} at depolarized potentials of a cell, preferably encoding a sodium channel and/or a functional equivalent of a sodium channel and/or a sodium channel with altered kinetics and/or an alpha subunit of a sodium channel and/or a beta-subunit of a sodium channel, and
at least one nucleic acid encoding a compound capable of increasing the firing frequency of a cell by increasing intracellular cAMP and/or by decreasing action potential duration.

57. A gene delivery vehicle or a vector according to claim 56, comprising a nucleic acid sequence or a functional equivalent thereof encoding an HCN channel, preferably HCN2, and a nucleic acid sequence or functional equivalent thereof encoding SkM1.

58. A cell according to claim 1, wherein said cell comprises a myocardial cell.

59. A cell according to claim 1, wherein said cell comprises a cardiac stem cell or cardiac progenitor cell.

60. A method according to claim 48, wherein said compound capable of diminishing electrical coupling between said cell and surrounding cells comprises a device comprising means for providing a cell with a compound capable of providing and/or increasing a pacemaker current I_{f}, and means for diminishing electrical coupling between said cell and surrounding cells.

61. A method according to claim 48, wherein said compound capable of diminishing electrical coupling between said cell and surrounding cells comprises an siRNA and/or antisense nucleotide sequence against connexin 43 and/or an siRNA and/or antisense nucleotide sequence against connexin 40 and/or a nucleic acid sequence encoding a compound selected from the group consisting of:
a compound capable of reducing the amount and/or activity of gap junction proteins connecting said cell and surrounding cells, a gap junction protein with a diminished conductor capacity as compared to connexin 43, a gap junction protein with a diminished conductor capacity as compared to connexin 40, a compound capable of reducing the amount and/or activity of connexin 43 of said cell, a compound capable of reducing the amount and/or activity of connexin 40 of said cell, a connexin with a lower conductor capacity than the conductor capacity of connexin 43, a connexin with a lower conductor capacity than the conductor capacity of connexin 40, connexin 30.2 or a functional equivalent thereof, connexin 45 or a functional equivalent thereof, connexin 43Δ or a functional equivalent thereof, a transcription factor capable of reducing connexin 43 expression, a transcription factor capable of reducing connexin 40 expression and TBX3 or a functional equivalent thereof.

62. A method according to claim 48, wherein said compound capable of providing and/or increasing a pacemaker current I_{f} comprises an siRNA and/or antisense nucleotide sequence against a phosphodiesterase and/or a nucleic acid sequence encoding a compound selected from the group consisting of: a cAMP producing enzyme, an adenylate cyclase, adenylate cyclase-1, adenylate cyclase-8, a compound capable of increasing the amount and/or activity of a cAMP producing enzyme, a compound capable of reducing the amount and/or activity of an enzyme involved with cAMP breakdown, and a phosphodiesterase with a diminished function as compared to wild type phosphodiesterase.

63. A method according to claim 48, wherein said compound capable of reducing the inward rectifier current I_{K1} of said cell comprises an siRNA and/or antisense nucleotide sequence against an inwardly-rectifying potassium channel and/or a nucleic acid sequence encoding a compound selected from the group consisting of: an inwardly-rectifying potassium channel with a diminished function as compared to the same kind of inwardly-rectifying potassium channel in a wild type form, and a Kir2.1 channel or a functional equivalent thereof.
